(19) United States
(12) Patent Application Publication
(10) Pub. No.: US 2024/0026370 A1
(43) Pub. Date: Jan. 25, 2024

(54) METHODS FOR THE IMPROVED RECOVERY OF SECRETED COMPOUNDS IN A HAIRY ROOTS-BASED EXPRESSION SYSTEM

(71) Applicant: SAMABRIVA, Amiens (FR)

(72) Inventors: Florian CARDON, Roiglise (FR); Camille LEMASSON, Amiens (FR); Marina GUILLET, Blain (FR)

(73) Assignee: SAMABRIVA, Amiens (FR)

(21) Appl. No.: 18/254,253

(22) PCT Filed: Nov. 25, 2021
(86) PCT No.: PCT/EP2021/083070

§ 371 Date: May 24, 2023

(30) Foreign Application Priority Data
Nov. 25, 2020 (EP) 20306443.1

(51) Int. Cl. C12N 15/82; C12N 9/24

(52) U.S. Cl. CPC C12N 15/8257; C12N 9/2402; C12Y 302/01076

(57) ABSTRACT

A method for recovering a secreted compound of interest in a hairy roots-based expression system by contacting the hairy roots with a solution including from about 250 mM to about 4 M of a salt. This method allows to increase the yield of recovery of the secreted compound of interest.

### FIELD OF INVENTION

The present invention relates to a method for recovering a secreted compound in a hairy roots-based expression system.

### BACKGROUND OF INVENTION

Hairy roots from plants have been widely studied and used for the production of specialized/secondary metabolites of industrial and pharmaceutical interest. Since the 1990s, the production of recombinant proteins has been considered as another promising application of hairy root cultures. This system presents numerous similarities with the one used to produce recombinant proteins from mammalian cell lines (such as, e.g., Chinese hamster ovary (CHO) cells, human cell lines, bacteria), among which the fact that the whole process is maintained under sterile conditions in a confined bioreactor.

However, this plant-based technology offers relevant and advantageous differences compared to the mammalian classical expression systems. As a matter of fact, the selected hairy root clones are easily grown in tailor-made optimal conditions in a simple culture medium with a better safety profile than media containing human- or animal-derived constituents. This process allows the production of compounds, such as recombinant proteins or valuable metabolites, in an axenic environment in at least 350 L bioreactors with a straightforward purification scheme that does not require a protein extraction phase as in common mammalian cells-based expression systems.

However, the recovery of recombinant proteins and valuable metabolites from such hairy roots-based expression systems necessitates several purification steps at the end of the process, including for example centrifugation steps. These purification steps are usually long processes, as they may take several days, and they may result in a substantial decrease in the yield of the recovered compound of interest, and may destroy the hairy roots and thus prevent any further new cycle of production. Consequently, there is a need to optimize the recovery of secreted compounds of interest, such as recombinant proteins or valuable metabolites, after their expression in a hairy roots-based expression system.

### SUMMARY

One aspect of the invention pertains to a method for recovering a secreted compound of interest in a roots-based expression system, in particular a hairy roots-based expression system, the method comprising contacting the roots with a solution comprising from about 250 mM to about 4 M of a salt (i.e., incubating the roots in a solution comprising from about 250 mM to about 4 M of a salt).

In some embodiments, the roots-based expression system comprises a phase of culture wherein the compound of interest is produced by the roots in a culture medium; optionally in the presence of an inductor of rhizocals.

In certain embodiments, the salt is organic or inorganic.

In some embodiments, the salt is selected from a group consisting of sodium (Na) salt, potassium (K) salt, chloride (Cl) salt, sulfate (SO₄) salt, nitrate (NO₃) salt, ammonium (NH₄) salt, phosphate (PO 4) salt, and any combination thereof. In some embodiments, the salt is selected from a group consisting of sodium (Na) salt, potassium (K) salt, chloride (Cl) salt, and any combination thereof.

In certain embodiments, the salt consists in sodium chloride (NaCl), potassium chloride (KCl), potassium nitrate (KNO₃), sodium sulfate (Na₂SO₄), potassium phosphate (KH₂PO₄), ammonium sulfate (NH₄)₂SO₄, ammonium nitrate (NH₄NO₃), sodium carbonate (Na₂CO₃), sodium glutamate (C₅H₈NNaO₄), sodium citrate (Na₃C₆H₅O₇), and/or sodium acetate (C₂H₃NaO₂). In certain embodiments, the salt consists in sodium chloride (NaCl), potassium chloride (KCl), sodium sulfate (Na₂SO₄), sodium glutamate (C₅H₈NNaO₄), sodium citrate (Na₃C₆H₅O₇), and/or sodium acetate (C₂H₃NaO₂).

In some embodiments, the solution of salt comprises from about 500 mM to about 2.5 M, preferably from about 500 mM to about 2 M, more preferably about 1M of salt.

In certain embodiments, the step of contacting the roots with the solution comprising the salt is performed for about 5 mM to about 24 h, preferably for about mM to about 18 h. In other words, in certain embodiments, the roots are incubated in the solution comprising the salt for about 5 mM to about 24 h, preferably for about 15 min to about 18 h.

In certain embodiments, the culture medium comprises less than 250 mM of salt, preferably a salt selected from a group consisting of a sodium (Na) salt, a potassium (K) salt, a chloride (Cl) salt, a sulfate (SO₄) salt, a nitrate (NO₃) salt, an ammonium (NH₄) salt, a phosphate (PO₄) salt or any combination thereof. In certain embodiments, the culture medium comprises less than 250 mM of salt, preferably a salt selected from a group consisting of a sodium (Na) salt, a potassium (K) salt, a chloride (Cl) salt, or any combination thereof.

In certain embodiments, said roots, in particular said hairy roots, belong to the species Brassica rapa rapa, Brassica napus, Salvia Milthiorrhiza, Panax Ginseng, Armoracia rusticana, Trigonella foenumgraceum, Lippia dulcis, Lithospermum erythrorhizon, Ophiorrhiza pumila, and Echinacea purpurea, Echinacea Angustifolia, Puerariaphaseoloides, Harpagophytum Procumbens, Morinda Citrifolia, Hypericum Perforatum, Derris trifolia, Salvia miltiorrhiza, Salvia prevalzkii, Echinacea pallida, Cistanche tubulosa, Glycyrrhiza glabra, Sophora flavescens, Rhodiola Rosea, Polygonum cuspidatum, Fallopia multiflora, Lepidium peruvianum, Whitania Somnifera, Astragalus Membranaceous, Berberis Vulgaris, Sanguinaria canadensis, Eleutherococcus Senticosus, Cannabis sativa, Hydrastis Canadensis, Arctium Majus, Piper methysticium, Pueraria lobata, Glycyrrhiza uralensis, Ptychopetalum olacoides, Dioscorea Vollosa, Yucca shidigera, Panax quinquefolium, Azadirachta indica, Catharanthus trichophyllus, Calystegia sepium, Atropa belladonna, Hyoscyamus muticus, Artemisia annua, Datura stramonium, Arabidopsis thaliana, Stizolobium, Hassjoo, Ipomea aquatica, Perilla fruitescnens, Catharanthus roseus, Taxus brevifolia, Gloriosa Superba, Saponaria officinalis, Solanum tuberosum, Nicotiana tabacum, Nicotiana benthamiana or Cinchosa Pubescens, preferably to the Brassica rapa rapa or Brassica napus species.

In some embodiments, said compound of interest is selected from the group consisting of metabolites, non-peptidic hormones and recombinant proteins.

In certain embodiments, the recombinant protein is selected from the group consisting of allergens; vaccines; enzymes; enzyme inhibitors; antibodies; antibody fragments; antigens, toxins; anti-microbial peptides; peptidic hormones; growth factors; blood proteins, in particular albumin, coagulation factors, transferrin; receptors and/or signaling proteins; protein component of biomedical standards; protein component of cell culture media; fusion and/or tagged proteins; cysteine (disulfide bridges)-rich peptides and proteins; and plant proteins, in particular lectins, papain.

In some embodiments, the metabolites are selected from the group consisting of polyphenols, alkaloids, cannabinoids, terpenoids, steroids, flavonoids, and tannins

Another aspect of the invention relates to a method for the continuous production of a secreted compound of interest by a roots-based expression system, in particular a hairy roots-based expression system, the method comprising:
a) the production of a compound by the roots in a culture medium, optionally in a culture medium suitable for rhizocals induction;
b) the removal of the culture medium;
c) the recovery of the secreted compound by contacting the roots with a solution comprising from about 250 mM to about 4 M of a salt, that is to say by incubating the roots in a solution comprising from about 250 mM to about 4 M of a salt;
d) optionally, the rinsing of the roots, preferably with water or fresh culture medium; and
e) the addition of a fresh culture medium to the roots as treated in step c) or d), wherein steps a) through e) are sequentially repeated between about 1 and about 5 times.

### Definitions

In the present invention, the following terms have the following meanings:

“About” preceding a figure encompasses plus or minus 10%, or less, of the value of said figure. It is to be understood that the value to which the term “about” refers to is itself also specifically, and preferably, disclosed.

“Comprise” is intended to mean “contain”, “encompass” and “include”. In some embodiments, the term “comprise” also encompasses the term “consist of”.

“Recover” is intended to mean that the product of interest, which is synthesized by the root system according to the invention, in particular the hairy root system, is physically separated from the root system itself. In some embodiments, the terms “recovery”, “elution” or “obtention” may be substituted to one another.

“Endogenous compound”, with regards to a roots-based expression system, in particular to a hairy roots-based expression system, refers to a compound which originates from the roots, that is to say which is naturally expressed by the roots, without any requirement for said roots to be genetically modified. Examples of endogenous compounds expressed by roots, in particular hairy roots, include metabolites and non-peptidic hormones.

“Secreted compound” refers to a compound which, upon synthesis in the cells of the root system, in particular of the hairy root system, crosses the cellular membrane/envelop and is to be localized outside these cells.

“Roots-based expression system” (also sometimes referred to as “root system” or “root expression system”) refers to a culture of vegetal roots, previously engineered so that they can synthesize a compound of interest, allowing in fine the roots to produce said compound.

Hairy roots-based expression system” (also sometimes referred to as “hairy root system” or “hairy root expression system”) refers to a culture of hairy roots, either previously engineered (for example genetically modified) or not, so that they can synthesize a compound of interest, allowing in fine the hairy roots to produce said compound.

“Contacting the roots with salt” refers here to the action to put in a same recipient the roots, in particular the hairy roots, and the solution comprising or consisting of salt in order to perform a washing of the roots. In other words, as used herein, contacting the roots with salt corresponds to incubating the roots, in particular the hairy roots, in a solution comprising or consisting of salt.

“Phase of culture” refers to the phase of culture of the roots, in particular the hairy roots, wherein the roots, in particular the hairy roots, are cultured within an appropriate culture medium in order to maintain the roots, in particular the hairy roots, in a state in which the cells of the roots, in particular of the hairy roots, may divide, have an active metabolism, increase their overall biomass and/or produce a compound of interest.

“Culture medium” refers to a solid or liquid medium containing all the required nutrients in which roots, in particular hairy roots, are cultivated.

“Rhizocal” or “rhizocallus” refers to a conic-shaped structure connected to the roots, in particular to the hairy roots, also termed lateral root emergence, which develops alongside of the roots in a solidarized way. In practice, rhizocals (or “rhizocalli”) may be induced in a culture of roots, in particular in a culture of hairy roots, by the addition in the culture medium of one or more agent(s) that promote(s) the induction of rhizocals such as, for example, the hormone called auxin or synthetic auxins such as 2,4-dichlorophenoxyacetic acid (2,4-D). The presence of “rhizocals” in a culture of hairy roots is often associated with a better yield in the production of a compound of interest by the hairy roots.

“Rhizocals induction” refers to a phase of culture of the roots, in particular of the hairy roots, wherein the culture medium is supplemented with an agent, for example a hormone, more specifically an auxin, such as, e.g., 2.4-D, which is capable of promoting a biomass growth cessation in the same time as an induction of rhizocals. The rhizocals, which correspond to a modification of the structure of the roots, enable an increase of the ability of the roots to secrete a compound of interest.

“Salt” refers to a chemical compound consisting of an ionic assembly of cations and anions. Salts are composed of equal amounts of cations (positively charged ions) and anions (negatively charged ions) so that the product is electrically neutral (without a net charge). The salt can be “inorganic”, meaning that the constitutive ions lack carbon-hydrogen bonds, such as NaCl, containing chloride ions (Cl⁻) and sodium ions (Nat). Alternatively, the salt can be “organic”, meaning that the anions or the cations contain carbon in covalent bonding, such as sodium acetate (C₂H₃NaO₂), containing acetate ions (CH₃COO⁻) and sodium ions (Nat).

“Stirring” refers to the action causing a slight movement of a solution/suspension in a recipient, preferably in a constant manner, in order to homogenize the distribution of the components in the solution/suspension.

“Metabolites” refers to small molecules i.e., intermediate or final products of metabolic reactions, such as, e.g., polyphenols, alkaloids, cannabinoids, terpenoids, steroids, flavonoids and tannins Valuable metabolites, i.e., metabolites of interest, may be naturally synthesized and secreted by roots, in particular by hairy roots. Alternatively, the synthesis and secretion of valuable metabolites may be artificially induced in roots, in particular in hairy roots (for example by adding an elicitor to the culture medium, by exposing the roots to a stress and/or by genetically modifying the metabolic pathway).

“Recombinant protein” refers to a protein encoded by a DNA nucleic acid that has been cloned in a vector system that supports expression of the corresponding DNA nucleic acid, including the transcription into a messenger RNA (mRNA) and translation of said messenger RNA into the protein. The expression “recombinant protein” or “protein of interest” are used interchangeably.

“Continuous production” refers to a roots-based expression system, in particular to a hairy roots-based expression system, which allows to achieve several cycles of production/synthesis and recovery of a compound of interest. Illustratively, upon recovery of the compound of interest, such as a protein of interest, by washing the roots with salt, the roots may be washed with water or culture medium and be treated so as to start another cycle of production/synthesis and recovery. In practice, a fresh culture medium is added to the washed roots and a new production of the compound of interest, such as a protein of interest, can be initiated. Continuous production may be performed according to the invention because the root biomass is not destroyed after each production/recovery cycle.

“Adventitious roots” refers to root emergences which appear during the physiological development of the plant and hence occurs naturally.

“Hairy root” refers to root emergences which appear after the infection of a plant by Rhizobium rhizogenes (previously referred to as Agrobacterium rhizogenes) bacteria or by Rhizobium radiobacter (also known as Agrobacterium Tumefaciens) bacteria harboring rol genes for example.

### DETAILED DESCRIPTION

The present invention focuses on a method for improving the recovery of a compound of interest produced in a roots-based expression system, in particular in a hairy roots-based expression system, such as, e.g., a hairy roots-based system obtained from Brassica rapa rapa or from Brassica napus. The inventors have discovered that performing a step of washing (or incubating) the hairy roots that have synthesized and secreted a compound of interest with a salt solution, such as, for example, NaCl, KNO₃ or C₂H₃NaO₂, at a concentration of at least 250 mM results in a significant improvement of the yield of recovery of the secreted compound of interest.

Without wanting to be bound to a theory, the inventors believe that the secreted compound of interest adhere to the roots, and that treatment with a salt “releases” the compound of interest in the culture medium, from which it can further be purified.

A first aspect of the invention relates to a method for recovering a secreted compound of interest in a roots-based expression system, in particular in a hairy roots-based expression system, the method comprising contacting the roots with a solution comprising from about 250 mM to about 4 M of a salt. In other words, a first aspect of the invention relates to a method for recovering a secreted compound of interest in a roots-based expression system, in particular in a hairy roots-based expression system, the method comprising incubating the roots in a solution comprising from about 250 mM to about 4 M of a salt.

In some embodiments, the method for recovering a secreted compound of interest in a roots-based expression system, in particular in a hairy roots-based expression system, comprises:
a phase of culturing roots, in particular hairy roots, in a culture medium, optionally in the presence of an inductor of rhizocals or under conditions enabling the production of endogenous compounds, wherein a compound of interest is produced and secreted by the roots, and
a phase of contacting the roots with a solution comprising from about 250 mM to about 4 M of a salt (that is to say a phase of incubating the roots in a solution comprising from about 250 mM to about 4 M of a salt);
   thereby allowing the recovery of the compound of interest produced and secreted by the roots.

Thus, in some embodiments, the method for recovering a secreted compound of interest in a roots-based expression system, in particular in a hairy roots-based expression system, comprises:
a) the production and the secretion of a compound of interest by a roots-based expression system by culturing roots in a culture medium, optionally in the presence of an inductor of rhizocals or under conditions enabling the production of endogenous compounds;
b) the removal of the culture medium; and
c) the recovery of the secreted compound of interest by contacting the roots with a solution comprising from about 250 mM to about 4 M of a salt (that is to say by incubating the roots in a solution comprising from about 250 mM to about 4 M of a salt).

In some embodiments, upon synthesis and secretion, the compound of interest is localized in the close vicinity of the roots, in particular outside the cells of the roots, and the roots are contacted with (or incubated in) a solution comprising from about 250 mM to about 4 M of a salt to recover the compound.

Root-based expression systems, in particular hairy root-based expression systems, have been abundantly described in the state of the art. In some embodiments, the root-based expression system, in particular the hairy root-based expression system, according to the invention may be known from the state of the art or be a system adapted or derived therefrom.

In some embodiments, the roots-based expression system, in particular the hairy roots-based expression system, may be obtained by the infection of a plant by a suitable bacterial or viral strain, preferably a bacterial strain of Rhizobium rhizogenes (formerly known as Agrobacterium rhizogenes). or strain of Agrobacterium Tumefaciens harboring rol genes. This bacterial strain may comprise a vector containing an expression cassette comprising a gene encoding a protein of interest.

Within the scope of the invention, the term “expression cassette” refers to a nucleic acid construct which can be introduced in a cell and which allows the expression of the gene comprised in the expression cassette. In practice, a suitable expression cassette may comprise a promotor, a nucleic acid encoding the protein of interest, a terminator, a signal peptide and optionally regulatory sequences that allow controlling the steps of transcription (e.g., polyA sequence) and/or translation.

In some embodiments, the promotor may be a viral promotor, in particular a viral promotor derived from a Brassicaceae plant-infecting virus. In some embodiments, the promotor may be an inducible promotor, i.e., chemical or physical inducible system (e.g., copper, steroid, alcohol, light), such as, for example, Tet repressor-based, tetracycline de-repressible; tTA-based, tetracycline inactivable; glucocorticoid receptor based, dexamethasone inducible; AlcR-based, ethanol inducible; Ecdysone receptor (EcR)-based, EcR agonist inducible; and estrogen receptor-based, β-estradiol inducible. In practice, a suitable promotor may be a constitutive Cauliflower Mosaic Virus (CaMV) 35S simple or double promotor or the Nos promotor.

In some embodiments, the expression cassette may comprise regulatory sequences. In some embodiments the regulatory sequence may be selected from the group comprising, or consisting of, a TMV Ω enhancer, consensus sequence, or transcriptional factor.

In certain embodiments, the regulatory sequence may be a TMV Ω enhancer.

In some embodiments, the expression cassette may comprise a polyadenylation signal that consists of multiple adenosine monophosphates. In practice, the expression cassette may also comprise a CaMV polyA sequence.

In some embodiments, the terminator sequence may comprise a sequence from Agrobacterium tumefaciens (i.e., T-nos, tmas, tocs, tORF25, ttml, tg7), from Solanum tuberosum (i.e., tpinII), from Pisum sativum (i.e., tE9) or from Glycine max (i.e., t7S). In certain embodiments, a suitable regulatory sequence may be a CaMV T35S terminator.

In some embodiments, the expression cassette may comprise a nucleic acid sequence encoding a signal peptide. Within the scope of the invention and as well-known from the state of the art, a “signal peptide” is a short peptide sequence which is, in most of the cases, present at the N-terminus part of a protein and necessary for the protein to cross the cell plasma membrane and therefore be secreted outside the cell.

In certain embodiments, the signal peptide may be the native signal peptide of the protein of interest. In another embodiment, said signal peptide may be derived from a Brassicaceae plant. In practice, a suitable signal peptide may be an Arabidopsis pectin methylesterase (PME) signal peptide, such as Arabidopsis At1g69940.

In some embodiments, the expression cassette is then cloned into an expression vector, such as, e.g., a plasmid. Typically, the expression vector may be a binary vector suitable for expression in a plant cell, such as the pRD400, pBIN19, pBINPlus or pCAMBIA binary vector.

In practice, pBIN19, pBINPlus and pCAMBIA binary vector may be commercially available from Addgene®.

In some embodiments, the plasmid may be a pRD400 plasmid.

Within the invention, the expression vector, e.g., the plasmid, may be incorporated into a competent bacterium by any one of the different processes known from the state of the art, such as bacterial transformation or electroporation.

As used herein, the term “competent” refers to a bacterium that has an increased ability to uptake an extra genomic nucleic acid into its cytoplasm. The skilled artisan is familiar with techniques for preparing competent bacteria (see, e.g., J. Sambrook and D. Russell, Molecular Cloning: A Laboratory Manual, 3^{rd} ed., Cold Spring Harbor Laboratory Press, Cold Spring Harbor, N. Y. (2001)).

In some embodiments, competent bacteria for bacterial transformation may be chemically competent cells, in particular calcium chloride treated bacteria.

Electroporation consists in the utilization of an electrical field in a solution comprising plasmids and bacteria in order to increase the permeability of the bacteria cell membrane allowing plasmids to be introduced in the bacteria. Suitable protocols may be found, e.g., in Green and Sambrook (Molecular Cloning, 4^{th} Edition, 2012, Cold Spring Harbor Laboratory Press).

Typically, infection of the roots, in particular of the hairy roots, by a bacterium is performed by contacting the bacterium with the roots which has been wounded beforehand, as previously described in the state of the art.

In some embodiments, the bacterium may be Rhizobium rhizogenes (formerly known as Agrobacterium rhizogenes), Rhizobium radiobacter (formerly known as Agrobacterium tumefaciens), or Rhizobium vitits (formerly known as Agrobacterium vitis). In practice, the bacterium used to infect the roots, in particular the hairy roots, may be Rhizobium rhizogenes (formerly known as Agrobacterium rhizogenes).

Many strains of Rhizobium rhizogenes (formerly known as Agrobacterium rhizogenes) can be used to perform the invention. Suitable strains include but are not limited to strain TR7 (or ATCC 25818 or LBA 9402), A4T, A4, ATCC 11325, LMG 155, LBA1334 and ATCC 15834.

In some embodiments, the strain of Rhizobium rhizogenes may be strain TR7 or strain ATCC 15834, preferably strain ATCC 15834.

In practice, the bacterium may be Rhizobium radiobacter (formerly known as Agrobacterium tumefaciens) harboring the rol genes, genetically integrated. Suitable strains include but are not limited to strain C₅₈, C₅₈C₁, LBA4404, GV2260, GV3100, A136, GV3101, GV3850, EHA101, EHA105 and AGL-1.

In some embodiments, the strain of Rhizobium radiobacter may be strain GV3101 or strain AGL-1, preferably strain GV3101.

The “rol genes” refers to the group of bacterial genes capable of inducing the formation of hairy roots and also able to affect growth and morphogenetic potential of plant cells, at least in part by altering the capability to respond to plant hormones. In some embodiments, the roots-based expression system, in particular the hairy roots-based expression system, achieves the production of a compound of interest.

According to some embodiments, the roots-based expression system, in particular the hairy roots-based expression system, is a system wherein roots are genetically modified and are used to produce a compound of interest such as a recombinant protein.

In a particular embodiment, a molecular construction has been evaluated for its ability to produce the recombinant protein of interest in high yield. This molecular construction is characterized by the use of a 35S double promoter, a TMV ω enhancer, a PME signal peptide, the nucleic acid encoding the recombinant protein and a 35S terminator.

In some embodiments, the whole sequence is codon-usage optimized for Brassica rapa rapa taking into account a GC content around 50-60%. The sequence is then gene-synthesized and cloned into, first, an intermediary plasmid (pUC plasmid), then into the binary plasmid pRD400. The sequencing of the pRD400 plasmid having integrated the molecular construction makes it possible to validate the integrity of the gene construct. The binary plasmid is then incorporated into competent R. rhizogenes bacteria by electroporation. Finally, the incorporation of the plasmid into the transformed R. rhizogenes clone is validated by DNA sequencing.

Plantlets of Brassica rapa rapa are then infected with this recombinant R. rhizogenes clone. The resulting clones are individualized and are all cultured in solid, then liquid culture medium. Antibiotics are only used for the 5 first cycles of culture and are only dedicated to eliminate R. rhizogenes. Apart from this very first step, all the process is antibiotic-free.

The first selection of the hairy root clones is based on their growth capacity. RNA is extracted from some of these hairy root clones and the integration of the gene encoding the protein of interest is confirmed by RT-PCR.

To refine and simplify the screening of the clones, a specific activity test is usually set up, as far as it is relevant (e.g., production of enzymes). This test is then applied on the clones to screen, as well as on samples generated during the different downstream steps. Using this activity assay, it is possible to identify the best producing clone(s).

According to some embodiments, the roots-based expression system, in particular the hairy roots-based expression system, is a system wherein roots are used to produce an endogenous compound of interest such as a metabolite or a non-peptidic hormone. In some embodiment, the roots used to produce an endogenous compound of interest, in particular the hairy roots, are not genetically modified. In some embodiment, the roots used to produce an endogenous compound of interest, in particular the hairy roots, are genetically modified. For example, roots, in particular hairy roots, may thus be used for the production of specialized/secondary metabolites of industrial interest. As used herein, “metabolites” refers to small molecules, i.e., intermediate or final products of metabolic reactions, such as, e.g., polyphenols, alkaloids, cannabinoids, terpenoids, steroids, flavonoids and tannins naturally expressed in plants.

In some embodiments, the production of an endogenous compound of interest is artificially induced in the roots-based expression system, in particular the hairy roots-based expression system, by culturing the roots, in particular the hairy roots, under conditions enabling the production of said endogenous compound of interest. Conditions enabling the production of endogenous compounds of interest in roots, in particular in hairy roots, are well-known in the art. Examples of conditions enabling the production of endogenous compounds of interest in roots, in particular in hairy roots, include adding an elicitor to the culture medium and exposing the roots to a stress.

In some embodiments, the production of an endogenous compound of interest is artificially induced in the roots-based expression system, in particular the hairy roots-based expression system, by adding an elicitor to the culture medium. In some embodiments, the production of an endogenous compound of interest is artificially induced in the roots-based expression system, in particular the hairy roots-based expression system, by exposing the roots, in particular the hairy roots, to a stress. Depending on the endogenous compound of interest to be produced, one skilled in the art will be able to determine the elicitor to be added to the culture medium or the stress to be applied to the roots, in particular to the hairy roots. Examples of elicitors include methyl jasmonate, jasmonic acid, chitosan, salicylic acid, jasmonate, cadmium chloride (CdCl₂), coumarine or furocoumarine, cyclodextrin, gibberellic acid, Phytopthora parasitica filtrate, Aspergillus niger, cellulase, and Bacteria sp. Examples of stress that can be applied to induce the production of an endogenous compound of interest include UV, such as UV-B.

In some embodiments, the elicitor added to the culture medium is selected from the group comprising or consisting of methyl jasmonate, jasmonic acid, chitosan, salicylic acid, jasmonate, cadmium chloride (CdCl₂), coumarine or furocoumarine, cyclodextrin, gibberellic acid, Phytopthora parasitica filtrate, Aspergillus niger, cellulase, and Bacteria sp. In some embodiments, the elicitor added to the culture medium is selected from the group comprising or consisting of methyl jasmonate, jasmonic acid, chitosan, and salicylic acid.

In some embodiments, the method is performed in a non-sterile environment.

In one alternative embodiment, the method is performed in a sterile environment. In practice, the vessels and/or the culture media may be sterilized according to the protocols known from the state of the art. Examples of sterilization treatments include heat-treatment (steam sterilization, high-temperature dry sterilization), UV treatment, and gamma ray treatment.

In some embodiments, the roots-based expression system, in particular the hairy roots-based system, comprises a phase of culture wherein the compound of interest is produced by the roots in a culture medium; optionally in the presence of an inductor of rhizocals. In other words, in some embodiments, the method first comprises a phase of culturing roots, in particular hairy roots, in a culture medium, wherein the compound of interest is produced and secreted by the hairy roots; optionally in the presence of an inductor of rhizocals.

In some embodiments, the roots-based expression system, in particular the hairy roots-based system, comprises a phase of culture wherein the compound of interest is produced by the roots in a culture medium; optionally under conditions enabling the production of an endogenous compound of interest. In other words, in some embodiments, the method first comprises a phase of culturing roots, in particular hairy roots, in a culture medium, wherein the compound of interest is produced and secreted by the hairy roots; optionally under conditions enabling the production of an endogenous compound of interest.

Thus, in some embodiments, the method for recovering a secreted compound of interest in a roots-based expression system, in particular in a hairy roots-based expression system, comprises:
a phase of culturing roots, in particular hairy roots, in a culture medium, wherein a compound of interest is produced and secreted by the roots; optionally in the presence of an inductor of rhizocals or under conditions suitable for the production of endogenous compounds; and
a phase of contacting the roots with (or incubating the roots in) a solution comprising from about 250 mM to about 4 M of a salt;
   thereby allowing the recovery of the compound of interest produced and secreted by the roots.

As used herein, the term “culture medium” is a substance containing nutrients in which roots, in particular hairy roots, can be maintained and/or grown. Culture media thus contain all the elements that the roots, in particular the hairy roots, need for survival and/or growth. An undefined medium may comprise a carbon source, water, salts, a source of amino acids and a source of nitrogen.

In practice, a suitable culture medium according to the invention may comprise (i) one or more pH buffering system(s); (ii) one or more inorganic salt(s); (iii) one or more trace element(s); (iv) one or more free amino acid(s); (v) one or more vitamin(s); (vi) one or more hormone(s); (vii) one or more carbon/energy source(s).

Culture media for roots-based expression systems are well known in the art. In some embodiments, a suitable medium, in particular for hairy roots-based expression systems, may be Standard Gamborg's (B5) medium, Murashige and Skoog's (MS) basal medium and N6 medium.

In some embodiments, germination and seedling growth may occur at a temperature ranging from about 15° C. to about 26° C., preferably from about 20° C. to about 24° C. and more preferably at 22° C. or 23° C. In some embodiments, germination and seedling growth may occur at a temperature ranging from about 20° C. to about 25° C.

In some embodiments, germination and seedling growth may occur in a light/dark photoperiod from about 13 h to about 18 h, preferably from about 15 h to about 17 h, and more preferably from about 16 h.

In some embodiments, the phase of culture is performed for at least 5, 6, 7, 8, 9, 11, 12, 13, 14, 15, 16, 17, 18, 19, or 20 days. It is understood that the duration of the phase of culture may depend on the size of the recipient in which the culture is performed, as larger recipients may necessitate longer duration of the phase of culture.

In some embodiments, the phase of culture is performed for about 5 days to about days. Within the scope of the invention, the expression “for about 5 days to about 60 days” encompasses 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 and 60 days.

In some embodiments, the phase of culture of the roots, in particular of the hairy roots, is performed in a culture medium, wherein the culture medium is Gamborg B5 medium further comprising at least one saccharide. In some embodiments, the at least one saccharide is selected in the group comprising or consisting of sucrose, glucose, fructose, mannose, xylose and ribose. In certain embodiments, the saccharide is incorporated in the Gamborg medium at a concentration of from about 0.1% (0.1 g/100 ml) to about 15% (15 g/100 ml), preferably from about 1% to about 5%, more preferably of about 3%.

In some embodiments, the culture medium may be Gamborg B5 medium with 3% sucrose.

In some embodiments, the culture medium may be renewed one or more times during the phase of culture of the roots, preferably by an identical culture medium, preferably by an identical volume.

In some embodiments, for the induction of rhizocals, an inductor of rhizocals is added to the culture medium after about 5 days to about 55 days of culture and preferably after about 14 days of culture. Within the scope of the invention, the expression “after about 5 days to about 55 days of culture” encompasses after about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 and 55 days of culture.

In some embodiments, the induction of rhizocals is performed for about 5 days to about 30 days, preferably from about 10 to about 25, more preferably for about 14 days or about 25 days. In some embodiments, the phase of induction of rhizocals is performed for at least 5 days.

In some embodiments, the induction of rhizocals is performed in a culture medium in the presence of an inductor of rhizocals, in particular an auxin.

Within the scope of the invention “an inductor of rhizocals” means that the addition of said inductor of rhizocals in the culture medium leads to the appearance of rhizocals which are lateral root emergences appearing on hairy roots. These rhizocals are able to produce the compound of interest in a higher quantity than hairy roots with no rhizocals.

In some embodiments, the inductor of rhizocals is a hormone. In some embodiments, said hormone is an auxin.

In some embodiments, the auxin may be selected from the group comprising or consisting of 2,4-dichlorophenoxyacetic acid (2,4-D), 3-indoleacetic acid (IAA), indole-3-butyric acid (IBA), 1-naphthaleneacetic acid (NAA), 2,4,5-trichlorophenoxyacetic acid (2,4,5-T), 2,3,5-triiodoacetic acid, 4-chlorophenoxyacetic acid, 2-naphthoxyacetic acid, 1-naphthylacetic acid, 4-amino-3,5,6-trichloropicolinic acid, 3,6-dichloro-2-methoxybenzoic acid (Dicamba), derivatives thereof and the likes. In a particular embodiment, the auxin is 2,4-dichlorophenoxyacetic acid (2,4-D).

In some embodiments, the culture medium comprises an auxin, in particular 2.4-D, in a concentration of from about 0.1 mg/L to about 10 mg/L. Within the scope of the invention, “about 0.1 mg/L to about 10 mg/L” encompasses 0.1, 0.2, 0.3, 0.4, 0.5, 0.6, 0.8, 0.9, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8 and 10.0 mg/L.

In some embodiments, the culture medium is Gamborg B5 medium with 3% sucrose and 1 mg/L of 2.4-D. The addition of an auxin, in particular 2.4-D, allows the cessation of the biomass growth simultaneously with the formation of rhizocals, leading to an increase of the ability of the roots to secrete the compound of interest. As used herein, the term “biomass”, as known in the art, refers to the total weight (amount) of living plants in a culture, expressed as weight per volume of culture (w/v). To measure the biomass, the roots are separated from the culture medium and they are weighted in an appropriate weighing scale and related to the initial culture volume. In some embodiments, the biomass may also be expressed as a dry biomass, the water contained in the roots being evaporated before weighing the roots. The dry biomass is expressed as a dry weight per volume of culture (dry w/v). In practice, evaporation of the water contained in the roots may be performed at a temperature of about 70° C., for about 24 h.

In practice, the weight of the biomass is expressed in gram (g) or kilogram (kg), whereas the volume is expressed in milliliter (mL) or liter (L).

When the roots-based system, in particular the hairy roots-based system, comprises an inducible promoter, the culture medium may comprise an effective amount of the corresponding inducer, such as, e.g., a tetracycline, a glucocorticoid (e.g., dexamethasone), an alcohol (e.g., ethanol), an estrogen (e.g., (3-estradiol).

In some embodiments, the roots-based expression system, in particular the hairy roots-based system, comprises:
a) a first phase of growth of the roots in a first culture medium; and
b) a second phase of production of the compound of interest in the culture medium, optionally in a culture medium suitable for rhizocalli induction or under conditions suitable for the production of endogenous compounds.

In other words, in some embodiments, the method first comprises a first phase of growing the roots in a first culture medium; and a second phase of producing the compound of interest in a second culture medium; optionally in the presence of an inductor of rhizocals or under conditions suitable for the production of endogenous compounds.

Thus, in some embodiments, the method for recovering a secreted compound of interest in a roots-based expression system, in particular in a hairy roots-based expression system, comprises:
a first phase of growing roots, in particular hairy roots, in a first culture medium;
a second phase of producing a compound of interest in a second culture medium; optionally in the presence of an inductor of rhizocals or under conditions suitable for the production of endogenous compounds; and
a third phase of contacting the roots with (or incubating the roots in) a solution comprising from about 250 mM to about 4 M of a salt;
   thereby allowing the recovery of the compound of interest produced and secreted by the roots.

In some embodiments, the first and the second culture medium are the same culture medium, for example Standard Gamborg's (B5) medium, Murashige and Skoog's (MS) basal medium, N6 medium, or a specifically developed medium. In other words, in some embodiments, the first phase of growing roots and the second phase of producing a compound of interest are performed in the same culture medium which is renewed at least once between the first phase and the second phase.

In some embodiments, the first and the second culture medium are different culture media, for example selected from Standard Gamborg's (B5) medium, Murashige and Skoog's (MS) basal medium, N6 medium and a specifically developed medium. In other words, in some embodiments, the first phase of growing roots and the second phase of producing a compound of interest are performed in different culture media, which may each be renewed at least once during the first phase and the second phase.

In some embodiments, the culture medium comprises less than 250 mM of salt, preferably a salt selected from a group consisting of a sodium (Na) salt, a potassium (K) salt, a chloride (Cl) salt, a sulfate (SO₄) salt, a nitrate (NO₃) salt, an ammonium (NH₄) salt, a phosphate (PO₄) salt, and any combination thereof. In some embodiments, the culture medium comprises less than 250 mM of salt, preferably a salt selected from a group consisting of a sodium (Na) salt, a potassium (K) salt, a chloride (Cl) salt, and any combination thereof. In some embodiments, the culture medium comprises less than 100 mM of salt, preferably a salt selected from a group consisting of a sodium (Na) salt, a potassium (K) salt, a chloride (Cl) salt, a sulfate (SO₄) salt, a nitrate (NO₃) salt, an ammonium (NH₄) salt, a phosphate (PO₄) salt, and any combination thereof. In some embodiments, the culture medium comprises less than 100 mM of salt, preferably a salt selected from a group consisting of a sodium (Na) salt, a potassium (K) salt, a chloride (Cl) salt, and any combination thereof.

In some embodiments, the culture medium comprises less than 250 mM of salt wherein said salt is a sodium (Na) salt, a potassium (K) salt, a chloride (Cl) salt, a sulfate (SO₄) salt, a nitrate (NO₃) salt, an ammonium (NH₄) salt, a phosphate (PO₄) salt or any combination thereof. In some embodiments, the culture medium comprises less than 250 mM of salt wherein said salt is a sodium (Na) salt, a potassium (K) salt, a chloride (Cl) salt, or any combination thereof. In some embodiments, the culture medium comprises less than 100 mM of salt wherein said salt is a sodium (Na) salt, a potassium (K) salt, a chloride (Cl) salt, a sulfate (SO₄) salt, a nitrate (NO₃) salt, an ammonium (NH₄) salt, a phosphate (PO₄) salt or any combination thereof. In some embodiments, the culture medium comprises less than 100 mM of salt wherein said salt is a sodium (Na) salt, a potassium (K) salt, a chloride (Cl) salt, or any combination thereof.

In some embodiments, the culture medium may comprise less than 250 mM of salt. In some embodiments, the culture may comprise 1, 5, 10, 15, 20, 25, 30, 35, 40, 45, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 220, 225, 230, 235, 240, 245 or 249 mM of salt. In some embodiments, the culture medium may comprise less than 100 mM of salt. In some embodiments, the culture may comprise 1, 5, 10, 15, 20, 30, 35, 40, 45, 50, 55, 60, 65, 70, 75, 80, 85, 90, 95 or 99 mM of salt.

In some embodiments, the culture medium may comprise no salt.

In some embodiments, the culture of the root-based system, in particular of the hairy root-based system, according to the invention is performed in a suitable recipient.

In some embodiments, the recipient is a bioreactor or an Erlenmeyer flask. In some embodiments the bioreactor is at least a 25 L, at least a 200 L bioreactor or at least a 350 L bioreactor.

In some embodiments, the phase of culturing roots, in particular hairy roots, in a culture medium, wherein a compound of interest is produced and secreted by the roots; optionally in the presence of an inductor of rhizocals or under conditions suitable for the production of endogenous compounds, is performed under bubble-oxygenation.

In some embodiments, the roots, in particular the hairy roots, are contacted in the same recipient with a solution comprising salt in order to facilitate the recovery of the produced compound of interest, such as a protein of interest. In other words, in some embodiments, the roots, in particular the hairy roots, are incubated in a solution comprising salt (that is to say the roots, in particular the hairy roots, are placed in a recipient comprising a solution comprising salt) in order to facilitate the recovery of the produced compound of interest, such as a protein of interest.

In some embodiments, the solution used for contacting the roots comprises from about 250 mM to about 4 M of a salt. Within the scope of the invention, the term “about 250 mM to about 4 M” includes 250 mM, 500 mM, 750 mM, 1.00 M, 1.25 M, 1.50 M, 1.75 M, 2.00 M, 2.25 M, 2.50 M, 2.75 M, 3.00 M, 3.25 M, 3.50 M, 3.75 M and 4.00 M.

In some embodiments, according to the present invention, the roots in particular the hairy roots, are washed or incubated or treated with a salt. In some embodiments, the roots are washed or incubated or treated with a salt after the phase of culture. In some embodiments, the salt is added after a complete removal of the culture medium and optionally a step of rinsing of the roots in particular the hairy roots, with water, or with fresh culture medium.

The salt can be organic or inorganic. In some embodiments, an inorganic salt may be sodium chloride (NaCl), potassium chloride (KCl), potassium nitrate (KNO₃) potassium phosphate (KH₂PO₄), ammonium sulfate (NH₄)₂SO₄, ammonium nitrate (NH₄NO₃), sodium carbonate (Na₂CO₃) and/or sodium sulfate (Na₂SO₄) and an organic salt may be sodium glutamate (C₅H₈NNaO₄), sodium citrate (Na₃C₆H₅O₇), and/or sodium acetate (C₂H₃NaO₂). In some embodiments, an inorganic salt may be sodium chloride (NaCl), potassium chloride (KCl) and/or sodium sulfate (Na₂SO₄) and an organic salt may be sodium glutamate (C₅H₈NNaO₄), sodium citrate (Na₃C₆HsO₇), and/or sodium acetate (C₂H₃NaO₂).

In some embodiments, the salt has an ionic strength ranging from about 0.1 mol/liter to about 25 mol/liter of a salt. In some embodiments, the solution comprising from about 250 mM to about 4 M of a salt has an ionic strength ranging from about 0.1 mol/liter to about 25 mol/liter of a salt. Within the scope of the invention, the term “about 0.1 mol/liter to about 25 mol/liter” includes 0.1 mol/liter, 0.5 mol/liter, 1 mol/liter, 1.5 mol/liter, 2 mol/liter, 2.5 mol/liter, 3 mol/liter, 3.5 mol/liter, 4 mol/liter, 4.5 mol/liter, mol/liter, 5.5 mol/liter, 6 mol/liter, 6.5 mol/liter, 7 mol/liter, 7.5 mol/liter, 8 mol/liter, 8.5 mol/liter, 9 mol/liter, 9.5 mol/liter, 10 mol/liter, 10.5 mol/liter, 11 mol/liter, 11.5 mol/liter, 12 mol/liter, 12.5 mol/liter, 13 mol/liter, 13.5 mol/liter, 14 mol/liter, 14.5 mol/liter, 15 mol/liter, 15.5 mol/liter, 16 mol/liter, 16.5 mol/liter, 17 mol/liter, 17.5 mol/liter, 18 mol/liter, 18.5 mol/liter, 19 mol/liter, 19.5 mol/liter, 20 mol/liter, mol/liter, 21 mol/liter, 21.5 mol/liter, 22 mol/liter, 22.5 mol/liter, 23 mol/liter, 23.5 mol/liter, 24 mol/liter, 24.5 mol/liter, and 25 mol/liter.

In some embodiments, the salt has an ionic strength ranging from about mol/liter to about 16 mol/liter of a salt. In some embodiments, the solution comprising from about 250 mM to about 2.5 M of a salt has an ionic strength ranging from about mol/liter to about 16 mol/liter of a salt. Within the scope of the invention, the term “about 0.1 mol/liter to about 16 mol/liter” includes 0.1 mol/liter, 0.25 mol/liter, mol/liter, 0.75 mol/liter, 1 mol/liter, 1.25 mol/liter, 1.5 mol/liter, 1.75 mol/liter, 2 mol/liter, 2.25 mol/liter, 2.5 mol/liter, 2.75 mol/liter, 3 mol/liter, 3.25 mol/liter, 3.5 mol/liter, 3.75 mol/liter, 4 mol/liter, 4.25 mol/liter, 4.5 mol/liter, 4.75 mol/liter, mol/liter, 5.25 mol/liter, 5.5 mol/liter, 5.75 mol/liter, 6 mol/liter, 6.25 mol/liter, 6.5 mol/liter, 6.75 mol/liter, 7 mol/liter, 7.25 mol/liter, 7.5 mol/liter, 7.75 mol/liter, 8 mol/liter, 8.25 mol/liter, 8.5 mol/liter, 8.75 mol/liter, 9 mol/liter, 9.25 mol/liter, 9.5 mol/liter, 9.75 mol/liter, 10 mol/liter, 10.25 mol/liter, 10.5 mol/liter, 10.75 mol/liter, 11 mol/liter, 11.25 mol/liter, 11.5 mol/liter, 11.75 mol/liter, 12 mol/liter, 12.25 mol/liter, 12.5 mol/liter, 12.75 mol/liter, 13 mol/liter, 13.25 mol/liter, 13.5 mol/liter, 13.75 mol/liter, 14 mol/liter, 14.25 mol/liter, 14.5 mol/liter, 14.75 mol/liter, 15 mol/liter, mol/liter, 15.5 mol/liter, 15.75 mol/liter, and 16 mol/liter.

Formula to determine the ionic strength of a salt solution are well-known in the art. For example, the ionic strength of a salt solution may be calculated with the following formula: I (or μ)=½Σᵢcᵢzᵢ², wherein I (or μ) is the ionic strength, c, is the molar concentration of ion i, and z, is the charge of ion i. Without wanting to be bound to a theory, the inventors hypothesize that an ionic strength ranging from about 0.1 mol/liter to about 25 mol/liter, in particular from about 0.1 mol/liter to about 16 mol/liter, is sufficient to significantly increased the yield of recovery of a compound of interest as illustrated hereinafter in the examples.

In some embodiments, the salt is selected from a group consisting of sodium (Na) salt, potassium (K) salt, chloride (Cl) salt, sulfate (SO₄) salt, nitrate (NO₃) salt, ammonium (NH₄) salt, phosphate (PO₄) salt, and any combination thereof. In some embodiments, the salt is selected from a group consisting of sodium (Na) salt, potassium (K) salt, chloride (Cl) salt, and any combination thereof.

In certain embodiments, the salt consists in sodium chloride (NaCl), potassium chloride (KCl), potassium nitrate (KNO₃), sodium sulfate (Na₂SO₄), potassium phosphate (KH₂PO₄), ammonium sulfate (NH₄)₂SO₄, ammonium nitrate (NH₄NO₃), sodium carbonate (Na₂CO₃), sodium glutamate (C₅H₈NNaO₄), sodium citrate (Na₃C₆H₅O₇), and/or sodium acetate (C₂H₃NaO₂). In certain embodiments, the salt consists in sodium chloride (NaCl), potassium chloride (KCl), sodium glutamate (C₅H₈NNaO₄), sodium sulfate (Na₂SO₄), sodium citrate (Na₃C₆H₅O₇), and/or sodium acetate (C₂H₃NaO₂). In certain embodiments, the salt consists in sodium chloride (NaCl), potassium nitrate (KNO₃), and/or sodium acetate (C₂H₃NaO₂).

In practice, the salt is NaCl, KCl, KNO₃ or C₂H₃NaO₂, as these salts are easily commercially available, at very low costs. In practice, the salt is NaCl or KCl, as these salts are easily commercially available, at very low costs.

In some embodiments, the solution of salt comprises from about 500 mM to about 2.5 M of salt. In some embodiments, within the scope of the invention, the term “about 500 mM to about 2.5 M” includes 500 mM, 750 mM, 1 M, 1.25 M, 1.5 M, 1.75 M, 2 M, 2.25 M and 2.5 M.

In some embodiments, the solution of salt comprises from about 500 mM to about 2 M of salt. In some embodiments, within the scope of the invention, the term “about 500 mM to about 2 M” includes 500 mM, 750 mM, 1 M, 1.25 M, 1.5 M, 1.75 M, and 2 M.

In some embodiments, the solution of salt comprises from about 500 mM to about 1.5 M of salt. In some embodiments, within the scope of the invention, the term “about 500 mM to about 1.5 M” includes 500 mM, 750 mM, 1 M, 1.25 M, and 1.5 M. In a preferred embodiment, the solution of salt comprises about 1 M of salt.

In some embodiments, the step of contacting the roots, in particular the hairy roots, with the solution comprising a salt (that is to say the step of incubating the roots, in particular the hairy roots, in the solution comprising a salt) is conducted at a temperature ranging from about 15° C. to about 26° C., preferably from about 20° C. to about 24° C. In some embodiments, the step of contacting the roots, in particular the hairy roots, with the solution comprising a salt (that is to say the step of incubating the roots, in particular the hairy roots, in the solution comprising a salt) is conducted at a temperature ranging from about 20° C. to about 25° C., and more preferably at 22° C. or 23° C.

In certain embodiments, the step of contacting the roots with the solution comprising the salt is performed for about 5 mM to about 24 h, preferably for about 15 min to about 18 h. In other words, in certain embodiments, the roots, in particular the hairy roots, are incubated in the solution comprising the salt for about 5 mM to about 24 h, preferably for about 15 mM to about 18 h. Within the scope of the invention, “for about mM to about 24 h” includes 5 min, 10 min, 20 mM, 30 mM, 1 h, 1.5 h, 2 h, 2.5 h, 3 h, 3.5 h, 4 h, 4.5 h, 5 h, 5.5 h, 6 h, 6.5 h, 7 h, 7.5 h, 8 h, 8.5 h, 9 h, 9.5 h, 10 h, 10.5 h, 11 h, 11.5 h, 12 h, 12.5 h, 13 h, 13.5 h, 14 h, 14.5 h, 15 h, 15.5 h, 16 h, 16.5 h, 17 h, 17.5 h, 18 h, 18.5 h, 19 h, 19.5 h, 20 h, 20.5 h, 21 h, 21.5 h, 22 h, 22.5 h, 23 h, 23.5 h and 24 h.

In certain embodiments, the step of contacting the roots, in particular the hairy roots, with the solution of salt is performed for about 5 mM to about 18 h, preferably for about 15 mM to about 16 h.

In some embodiments, the step of contacting the roots, in particular the hairy roots, with a solution comprising from about 250 mM to about 4 M of salt, preferably from about 500 mM to about 2.5 M of salt, may be performed without stirring. In other words, in some embodiments, the incubation of the roots, in particular of the hairy roots, in a solution comprising from about 250 mM to about 4 M of a salt, preferably from about 500 mM to about 2.5 M of a salt, may be performed without stirring.

In some embodiments, the step of contacting the roots, in particular the hairy roots, with a solution comprising from about 250 mM to about 4 M of a salt is performed under stirring.

Stirring refers to the action causing a slight movement of a solution/suspension in a recipient, preferably in a constant manner, in order to homogenize the distribution of the components in the solution/suspension.

In some embodiments, as known in the art, the stirring may be performed by mechanic or magnetic means. In some embodiments, the stirring may be performed by placing the recipient which contains the roots within the solution of salt on a constant moving support (e.g., a shaking table or an orbital shaker). Stirring performed by placing the recipient which contains the roots within the solution of salt on a constant moving support is also referred to as shaking. In some embodiments, the stirring is performed with the means of a magnet in the recipient containing the roots within the solution of salt and a magnetic agitator on which the recipient is placed.

In practice, the stirring may be performed using mechanical means and more particularly by placing the recipient which contains the roots within the solution of salt on a constant moving support, such as a shaking table or an orbital shaker.

In some embodiments, the stirring, in particular the shaking, is performed at a speed of from about 10 rpm to about 350 rpm, preferably at a speed of from about 10 rpm to about 300 rpm, more preferably at a speed of from about 10 rpm to about 100 rpm. Within the scope of the invention the expression “about 10 rpm to about 300 rpm” encompasses 10, 25, 50, 75, 100, 125, 150, 175, 200, 225, 250, 275, 300, 325 and 350 rpm. Within the scope of the invention the expression “about 10 rpm to about 300 rpm” encompasses 10, 25, 50, 75, 100, 125, 150, 175, 200, 225, 250, 275 and 300 rpm. Within the scope of the invention the expression “about 10 rpm to about 100 rpm” encompasses 10, 15, 20, 25, 30, 35, 40, 45, 50, 55, 60, 75, 80, 85, 90, 95 and 100 rpm.

In some preferred embodiments, the stirring is a gentle stirring. In some embodiments, a gentle stirring is a gentle shaking. In some embodiments, a gentle stirring is a gentle shaking performed at a speed of from about 10 rpm to about 100 rpm. In some embodiments, a gentle stirring is performed with no shaking and only by oxygenation of the culture, for example by bubble-oxygenation.

In some preferred embodiments, the step of contacting the roots, in particular the hairy roots, with a solution comprising from about 250 mM to about 4 M of salt is performed without shaking. In other words, in some preferred embodiments, the incubation of the roots, in particular of the hairy roots, in a solution comprising from about 250 mM to about 4 M of a salt is performed without shaking.

In some embodiments, the stirring is performed by oxygenation of the culture, namely, by the means of providing an oxygen flux to the culture. Examples of means of providing an oxygen flux to the culture include bubble-oxygenation, such as obtained with a sparger. In certain embodiments, the stirring is performed by recirculation of culture medium within the recipient, in particular by the means of an inner/outer flux of culture medium to/from the recipient.

In some embodiments, the step of contacting the hairy roots with a solution comprising from about 500 mM to about 4 M of salt is not followed by a centrifugation step. In other words, in some preferred embodiments, the incubation of the roots, in particular of the hairy roots, in a solution comprising from about 250 mM to about 4 M of a salt is not followed by any centrifugation step.

In some embodiments, the washing step with a salt (i.e., the step of incubating the roots, in particular the hairy roots, with the solution comprising a salt) allows to increase the yield in comparison to a method without any washing step.

As used herein, the term “yield”, as known in the art, refers to the amount of compound of interest, such as a protein of interest, recovered upon production and purification.

In some embodiments, the washing step allows to increase the yield from about 10% to about 100%. Within the scope of the invention “from about 10% to about 100%” includes 10, 15, 20, 25, 30, 35, 40, 45, 50, 55, 60, 65, 70, 75, 80, 85, 90, 95 and 100%.

The efficacy of recovery of the compound of interest may generally be measured by any suitable means for detecting said compound. Examples of suitable means for detecting a compound of interest may be ELISA, Western Blotting, immunoprecipitation, mass spectrometry, fluorescence, enzymatic assay, flow cytometry and the likes.

For example, in some embodiments, to evaluate the recovery of an enzyme, the appropriate substrate is added to a sample of the washing solution in order to evaluate the enzyme activity and/or production and then the recovery of the enzyme. In practice, a sample of the washing solution may be contacted with the appropriate substrate of the recovered enzyme and the activity and/or production of said enzyme may be monitored in suitable conditions. Illustratively, for an enzyme such as alpha-L-iduronidase (IDUA), enzymatic activity will be measured by adding the fluorogenic substrate sodium 4-methylumbelliferyl-α-L-Iduronide (4MU-I) into the washing solution and fluorescence will be evaluated using a plate reader.

In some embodiments, to evaluate the recovery of a natural fluorescent protein, a plate reader may be used. In particular, for evaluating the recovery of eGFP a plate reader may be used.

In certain embodiments, the roots are selected from a group consisting of adventitious roots, hairy roots, rhizocals, and any combination thereof, preferably, the roots are hairy roots.

In some embodiments, the roots can be adventitious roots, hairy roots, and/or rhizocals.

In some preferred embodiments, the roots are hairy roots. In some embodiments, the roots are hairy roots without rhizocals. In some embodiments, the roots are hairy roots with rhizocals.

In some embodiments, the roots, in particular the hairy roots, belong to the species Brassica rapa rapa, Brassica napus, Salvia Milthiorrhiza, Panax Ginseng, Armoracia rusticana, Trigonella foenumgraceum, Lippia dulcis, Lithospermum erythrorhizon, Ophiorrhiza pumila, and Echinacea purpurea, Echinacea Angustifolia, Puerariaphaseoloides, Harpagophytum Procumbens, Morinda Citrifolia, Hypericum Perforatum, Derris trifolia, Salvia miltiorrhiza, Salvia prevalzkii, Echinacea pallida, Cistanche tubulosa, Glycyrrhiza glabra, Sophora flavescens, Rhodiola Rosea, Polygonum cuspidatum, Fallopia multiflora, Lepidium peruvianum, Whitania Somnifera, Astragalus Membranaceous, Berberis Vulgaris, Sanguinaria canadensis, Eleutherococcus Senticosus, Cannabis sativa, Hydrastis Canadensis, Arctium Majus, Piper methysticium, Pueraria lobata, Glycyrrhiza uralensis, Ptychopetalum olacoides, Dioscorea Vollosa, Yucca shidigera, Panax quinquefolius, Azadirachta indica, Catharanthus trichophyllus, Calystegia sepium, Atropa belladonna, Hyoscyamus muticus, Artemisia annua, Datura stramonium, Arabidopsis thaliana, Stizolobium, Hassjoo, Ipomea aquatica, Perilla fruitescnens, Catharanthus roseus, Taxus brevifolia, Gloriosa Superba, Saponaria officinalis, Solanum tuberosum, Nicotiana tabacum, Nicotiana benthamiana and Cinchosa Pubescens, preferably to the Brassica rapa rapa and Brassica napus species.

In some embodiments, the roots, in particular the hairy roots, belong to a family selected in a group consisting of the Brassicaceae family, preferably the roots are of the Brassica rapa and Brassica napus species; the Solanaceae family; the Cannabaceae family; the Caryophyllaceae family; and the Vitaceae family.

In some embodiments, the roots, in particular the hairy roots, belong to the Brassicaceae family. In some embodiments, the roots, in particular the hairy roots, belong to the Brassicaceae family and are selected from the group of species consisting of Raphanus sativus, Raphanus sativus var. niger, Brassica Oleracea L. Convar, Brassica rapa, Brassica napus and Arabidopsis thaliana.

In a preferred embodiment, the roots, in particular the hairy roots, are Brassica rapa rapa roots or Brassica napus roots. In a preferred embodiment, the roots, in particular the hairy roots, are Brassica rapa rapa roots.

In some embodiments, the compound of interest is an endogenous compound or an exogenous compound.

In some embodiments, the compound of interest is selected from the group consisting of metabolites, non-peptidic hormones and recombinant proteins.

In some embodiments, the compound of interest obtained by the method of the invention has a purity of at least 20%, preferably at least 25%, more preferably 30, 40, 60, 70, 80, 90 or 95%.

In some embodiments, the compound of interest is a protein (also referred to as protein of interest).

In some embodiments, the protein of interest according to the invention is not naturally produced by the roots-based system, in particular by the hairy roots-based system.

In some embodiments, the compound of interest is a recombinant protein (also referred to as protein of interest).

In certain embodiments, the recombinant protein is selected from the group consisting of allergens; vaccines; enzymes; enzyme inhibitors; antibodies; antibody fragments; antigens, toxins; anti-microbial peptides; peptidic hormones; growth factors; blood proteins, in particular albumin, coagulation factors, transferrin; receptors and/or signaling proteins; protein component of biomedical standards; protein component of cell culture media; proteins assembled to form viruses, fusion and/or tagged proteins; cysteine (disulfide bridges)-rich peptides and proteins; and plant proteins, in particular lectins, papain.

In some embodiments, the protein of interest (or recombinant protein) is a protein from an animal species, preferably a mammalian species such as a primate, a canine, a feline, a rodent or an equine species. In some embodiments, the protein of interest (or recombinant protein) is a human protein.

In some embodiments, the protein of interest recovered according to the invention may be a glycosylated protein. As used herein, the term “glycosylated protein” refers to the result of the enzymatic process that attaches glycans to proteins. The glycosylation is a post-translational modification and glycans play a structural and functional role in membrane and secreted proteins.

As well known in the art, the most common glycosylation are the N-glycosylation and the O-glycosylation. The N-glycosylation refers to the addition of an oligosaccharide harboring a N-acetyl-glucosamine on an asparagine (Asn) amino acid included in the following sequence of a protein Asn-Xaa-Ser/Thr, Xaa being any amino acid except proline (Pro), serine (Ser) or threonine (Thr). The O-glycosylation refers to the addition of glycans to an —OH residue of some Ser and Thr amino acids of proteins.

In some embodiments, the compound of interest is an endogenous compound. In some embodiments, the compound of interest is a metabolite or a non-peptide hormone (also referred herein as non-peptidic hormone).

In some embodiments, the compound of interest is a metabolite. In certain embodiments, the metabolite is selected from the group consisting of polyphenols, alkaloids, cannabinoids, terpenoids, steroids, flavonoids, and tannins.

As used herein, “polyphenols, alkaloids, cannabinoids, terpenoids, steroids, flavonoids, and tannins” also encompass derivatives of these compounds. In practice, the term “derivatives” refers to compounds that share similar structures to their counterpart and have similar functions.

In some embodiments, the compound of interest is a non-peptide hormone (also referred herein as non-peptidic hormone).

Another aspect of the invention relates to a method for the continuous production of a secreted compound of interest as described hereinabove by a roots-based expression system, the method comprising:
a) the production of a compound of interest by the roots in a culture medium as described hereinabove, optionally in a culture medium suitable for rhizocals induction (i.e., in a culture medium comprising an inductor of rhizocals as described hereinabove) or under conditions suitable for the production of endogenous compounds (i.e., in a culture medium comprising for example an elicitor such as methyl jasmonate);
b) the removal of the culture medium;
c) the recovery of the secreted compound of interest by contacting the roots with (or incubating the roots in) a solution comprising from about 250 mM to about 4 M of a salt as described hereinabove;
d) optionally, the rinsing of the roots, preferably with water or fresh culture medium; and
e) the addition of a fresh culture medium to the roots as treated in step c) or d), wherein steps a) through e) are sequentially repeated between about 1 and about 5 times.

In some embodiments, the invention relates to a method for the continuous production of a secreted compound of interest as described hereinabove by a hairy roots-based expression system, the method comprising:
a) the production and the secretion of a compound of interest by hairy roots in a culture medium as described hereinabove, optionally in the presence of an inductor of rhizocals as described hereinabove or under conditions suitable for the production of endogenous compounds;
b) the removal of the culture medium;
c) the recovery of the secreted compound of interest by contacting the hairy roots with (or incubating the hairy roots in) a solution comprising from about 250 mM to about 4 M of a salt as described hereinabove;
d) optionally, the rinsing of the hairy roots, preferably with water or fresh culture medium; and
e) the addition of a fresh culture medium to the hairy roots as treated in step c) or d),
wherein steps a) through e) are sequentially repeated between about 1 and about 5 times.

In other words, in some embodiments, the invention relates to a method for the continuous production of a secreted compound of interest by a hairy roots-based expression system, the method comprising:
a) culturing hairy roots in a culture medium as described hereinabove, optionally in the presence of an inductor of rhizocals as described hereinabove or under conditions suitable for the production of endogenous compounds, wherein a compound of interest is produced and secreted by the hairy roots;
b) removing the culture medium;
c) recovering the secreted compound of interest by contacting the hairy roots with (or incubating the hairy roots in) a solution comprising from about 250 mM to about 4 M of a salt as described hereinabove;
d) optionally, rinsing the hairy roots, preferably with water or fresh culture medium; and
e) adding fresh culture medium to the hairy roots as treated in step c) or d), thereby starting a new culture of the hairy roots in culture medium, wherein steps a) through e) are sequentially repeated between about 1 and about 5 times.

In some embodiments, the method for continuous production refers to a method wherein the roots, in particular the hairy roots, are incubated in a culture medium at step a) for a phase of culture lasting from about 5 days to about 60 days. Within the scope of the invention, the expression “for about 5 days to about 60 days” encompasses 5, 6, 7, 8, 9, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 and 60 days.

In some embodiments, the step a) is performed for at least 5, 6, 7, 8, 9, 10, 11, 12, 13, 14, 15, 16, 17, 18, 19 or 20 days, preferably for at least 10 days, more preferably for at least 14 days.

In some embodiments, the culture medium is Gamborg B5 medium with 3% sucrose.

In some embodiments, for the induction of rhizocals, an inductor of rhizocals as described hereinabove is added to the culture medium after about 5 days to about 20 days of culture, preferably after about 10 days of culture, and more preferably after about 14 days of culture.

In some embodiments, the culture medium is Gamborg B5 medium with 3% sucrose and 1 mg/L of 2.4-D.

In some embodiments, the step b) consists in the removal of the culture medium before the recovery of the secreted compound c).

In some embodiments, the quantity (or concentration) of salt used at step c) allows to reuse the roots, in particular the hairy roots, for a new cycle of production after the recovery of the compound. In some embodiments, step c) consists in recovering the secreted compound of interest by contacting the hairy roots with (or incubating the hairy roots in) a solution comprising from about 500 mM to about 2.5 M, preferably from about 500 mM to about 2 M, of a salt as described hereinabove.

In some embodiments, after step c) there is optionally a step d) of rinsing of the roots, in particular the hairy roots, with a solution, preferably wherein the solution is water. In some embodiments, step d) is performed with a solution consisting of fresh culture medium. The optional step d) may be performed so as to “reset” the system, i.e., remove unconsumed nutrients and/or metabolic by-products synthesized during a cycle of growth and production.

In some embodiments, rinsing is carried out with water. In some embodiments, the water is sterile and preferably filtered, and more preferably demineralized. In certain embodiments, the water is ultrapure water.

In some embodiments, at step e) a fresh medium is added to the roots, in particular to the hairy roots, as treated in step c) or d) and the process through steps a) to e) is repeated between about 1 and about 5 times.

In some particular embodiments, in order to carry out this production in a continuous system, the following method is used. Before inoculation, the bioreactor is sterilized, preferably autoclaved. First, under the laminar hood, hairy roots are collected from a Working Transgenic Bank (Erlenmeyer flask). Total hairy roots are then weighed. These steps are carried out in sterile conditions. Then, under the laminar hood, the bioreactor is gently opened and the hairy roots are put inside the bioreactor, with the clamp. The bioreactor is then closed. A fresh culture medium is then injected in the bioreactor trough the liquid filter to maintain a sterile environment. When the bioreactor is filled, the stand is carried under the laminar hood. The liquid filter is taken off and replaced by a sealing cap. 500 μL of sterile antifoam is then added through one of the various inlets using a sterile syringe. Air inlet is then connected to the pressurized air network and air flowrate is set. This step is classically disclosed in the specialized literature.

After this inoculation phase, biomass growth is maintained for 14 days. At day 14, the culture medium is totally removed and replaced by a fresh culture medium in which a very small amount of 2.4-D (1 mg/L) is added. This auxin leads to the biomass growth cessation in the same time as the induction of rhizocals which correspond to a modification of the structure of the hairy roots enabling an increase of the ability of the hairy root to secrete the compound of interest, such as a protein of interest (the viability of the biomass remains effective). This strategy is described in the patent application PCT/FR2016/051149.

After about 10-14 days of culture of the hairy roots with rhizocals, a first sampling of the proteins can be carried out through a complete removal of the culture medium. The compound of interest, such as a protein of interest attached to the hairy roots, can be recovered using a 1M NaCl washing step (there is a 10 to 100-fold difference in compound yield between the washing solution and the culture medium). After rinsing with water or fresh culture medium, a fresh culture medium is reintroduced into the bioreactor and a new production can be initiated for 10-14 additional days. This cycle (from the removal of the culture medium and the addition of a fresh culture medium enriched in 2.4 D to the NaCl washing step) can be reproduced for several months.

In some embodiments, washing solutions are collected, the hairy roots are rinsed with ultrapure water and dried at 70° C. for 24 h for dry weight determination.

As illustrated in the example section hereinafter, the inventors have surprisingly shown that a significant increase in the yield of recovery of a compound of interest secreted in a hairy roots-based expression system can be obtained by incubating the hairy roots in a solution comprising from about 250 mM to about 4 M of salt. The inventors have shown that such a yield increase can be obtained using either an inorganic salt (such as sodium chloride (NaCl) or potassium nitrate (KNO₃)) or an organic salt (such as sodium acetate (C₂H₃NaO₂)). Moreover, the inventors have shown that the yield increase is observed using different species of hairy roots (such as, for example, Brassica rapa rapa or Brassica napus).

Strikingly, the increase in yield can be obtained without any stirring, in particular without any shaking. Furthermore, no centrifugation is required at all to recover a significant amount of the secreted compound of interest. Indeed, the inventors have been able to significantly increase the yield of recovery by incubating the hairy roots for as short as 15 min in a solution comprising for example 500 mM, 1 M or 2 M of a salt such as sodium chloride (NaCl). The method for recovering a secreted compound of interest in a hairy roots-based expression system as described herein thus presents the additional advantage of preserving the integrity of the hairy roots. In particular, it makes it possible to conduct several cycles of growth and production using the same hairy roots, for example in a method for the continuous production of a secreted compound of interest as described herein, with a significant amount of the secreted compound of interest being recovered at the end of each cycle of growth and production.

### BRIEF DESCRIPTION OF THE DRAWINGS

FIG. 1 represents different conditions of NaCl washing. (FIG. 1A) Fifteen cultures of a hairy roots clone of Brassica rapa rapa were grown during 14 days followed by an additional 25-day period of culture after the induction of rhizocals. Hairy roots were then washed according to the scheme. After different conditions of washing, the hairy roots dry weight was measured (FIG. 1B). rIDUA (recombinant alpha-L-iduronidase) production in culture medium after different conditions of washing was measured by enzymatic assay (FIG. 1C).

FIG. 2 represents the effect of stirring (i.e., shaking) on protein recovery. Fifty cultures of one clone of Brassica rapa rapa were grown during 14 days and then, after additional days of culture after induction of rhizocals, cultures were harvested and washed with NaCl using different protocols as indicated on the graphs. Hairy roots dry weight (FIG. 2A) and rIDUA production (FIG. 2B) were assessed.

FIG. 3 represents washing conditions using different salts. Cultures of Brassica napus hairy roots were grown during 10 days followed by an additional 10-day period of culture after the induction of rhizocals. Hairy roots were then washed with sodium chloride NaCl (0 and 100 rpm), sodium acetate or NaAc C₂H₃NaO₂ (100 rpm) and potassium nitrate KNO₃ (100 rpm). rIDUA production (FIG. 3A) and the ratio rIDUA/total protein (FIG. 3B) were then assessed; a and b represent statistical similarity (p=0.05) and * represents p≤0.05 based on Student test.

FIG. 4 represents the effect of the presence of NaCl in the culture medium. (FIG. 4A) Culture conditions are presented in the scheme. NaCl was added or not to the culture medium during the culture at a concentration of 100 mM and then the hairy roots were harvested and washed or not with NaCl at the indicated concentrations. Hairy roots dry weight (FIG. 4B) and total rIDUA production (FIG. 4C) of the washing solution were assessed in the different conditions.

FIG. 5 represents the effect of NaCl on protein recovery. (FIG. 5A) At the end of the culture of different Brassica rapa rapa hairy roots clones, the culture medium has been collected and the hairy roots have been washed with a 1 M NaCl solution (same volume as the culture medium) for 15 min. Then the rIDUA production has been measured both in the culture medium (CM) and in the NaCl washing solution (WS) of different clones. (FIG. 5B) Western Blot analysis showing the quantification of obtained rIDUA both in CM and in WS of one representative clone.

FIG. 6 represents the hairy roots continuous production system of eGFP. (FIG. 6A) Culture conditions are presented in the scheme. After washing with 1 M NaCl during 15 min, the hairy roots were rinsed and a fresh culture medium was added to initiate a novel production phase during 14 days. Dry weight (FIG. 6B) and eGFP production (FIG. 6C) of the washing solution were assessed in two successive cycles of harvest.

### EXAMPLES

The present invention is further illustrated by the following examples.

### Example 1: Effect of Salt Washing on the Recovery of Secreted rIDUA (Recombinant alpha-L-iduronidase)

1. Material and Methods

1.1. Molecular Construction

The molecular construction is characterized by the presence of a 35S double promoter, a TMV ω enhancer, a PME signal peptide, the nucleic acid encoding the recombinant protein IDUA and a 35S terminator.

1.2. Cultures of Hairy Roots

Cultures of a hairy root clone of Brassica rapa rapa producing rIDUA were grown for 14 days on Gamborg B5 medium with 3% of sucrose. At the end of the growth phase, the culture media were replaced by a fresh one, supplemented with 1 mg/L of 2,4-D to induce rhizocals. After 25 days, cultures were collected.

Cultures of a hairy root clone of Brassica napus IDUA-50 were grown for days on Gamborg B5 medium supplemented with 3% of sucrose. At the end of the growth phase, the culture media were replaced by a fresh one, supplemented with 1 mg/L of 2,4-D to induce rhizocals. After 10 days, cultures were collected.

1.3. Stirring Protocol

Forty cultures of one Brassica rapa rapa clone were cultured with a typical production process (i.e., 14 days of growth and 25 days of protein production after rhizocals induction). At the end of the process, cultures were harvested and washed (i.e., incubated in a washing salt solution) following different methods, as presented in the Table 1 below. Stirring (i.e., shaking) was performed at 40 rpm with a shaking table (New Brunswick®). The incubations with a salt solution (NaCl solution) were conducted at a temperature of 23° C.

TABLE 1

| Number of replicates | NaCl (M) | Washing time | | Stirring (40 rpm) |
|---|---|---|---|---|
| 10 | 1 | 15 | min | YES |
| 10 | 1 | 4 | h | YES |
| 10 | 1 | Overnight | | YES |
| 5 | 1 | 15 | min | NO |

| | | | |
|---|---|---|---|
| 5 | 1 | Overnight | NO |

The efficiency of each method was evaluated measuring hairy roots dry weight and the rIDUA production in the washing solution.

Cultures of one Brassica Napus clone were cultured with a typical production process (i.e., 10 days of growth and 10 days of protein production after rhizocals induction). At the end of the process, cultures were harvested and washed (i.e., incubated in a washing salt solution) following different methods as presented in the Table 2 below. Stirring (i.e., shaking) was performed at 100 rpm with an orbital shaker (New Brunswick Innova 2100). The incubations with the indicated salt solutions were conducted at a temperature of 23° C.

TABLE 2

| Number of | | | |
|---|---|---|---|
| replicates | Salt and concentration | Washing time | Stirring (rpm) |
| 3 | NaCl 1M | 15 min | 0 |
| 3 | | | 100 |
| 3 | KNO₃ 1M | | 100 |
| 3 | C₂H₃NaO₂ 1M | | 100 |

1.4. Collect and Protein Recovery

At the end of the protein production process by hairy roots, culture media were harvested and the hairy roots were washed (i.e., incubated) with the indicated concentrations of NaCl, KNO₃ or C₂H₃NaO₂ for a length of time ranging from 15 min to overnight, at a temperature of 23° C. Washing solutions were collected, the hairy roots were rinsed with ultrapure water and dried at 70° C. for 24 h for dry weight determination. rIDUA production in culture medium and washing solution was then determined by enzymatic assay.

1.5. rIDUA Detection by Western Blot

Samples (crude culture media) were resolved in AnykD mini protean TGX polyacrylamide gels (Bio-Rad®, Hercules, California). For Western blot analysis, proteins were transferred to nitrocellulose membranes (Bio-Rad®) using the Bio-Rad Turbo Trans-Blot system. The membranes were blocked in 5% fat-free milk (Blotting grade blocker, Bio-Rad®) in TBS buffer, incubated with a 1:1000 dilution of the mouse anti-a-L-Iduronidase (ABIN603316 from Antibodies-online®) followed by a 1:5000 dilution of a goat anti-mouse IgG-HRP antibody (sc-2005 from Santa Cruz Biotechnology®, Dallas, US). Staining was developed using Western Clarity ECL revelation kit (170-5060, Bio-Rad®).

1.6. Determination of the Human rIDUA Production

Turnip and Brassica Napus hairy root culture media collected from transformed hairy root cultures were used to determine the production of the recombinant protein of interest by using the fluorogenic substrate sodium 4-methylumbelliferyl-a-L-Iduronide (4MU-I) (Santa Cruz Biotechnology®) as described in (Ou et al., 2014; Mol. Genet. Metab. 111, 113-115). The 4MU-I substrate was diluted to a working solution of 400 μM 4MU-I with the reaction buffer 0.4 M sodium formate, pH 3.5. Twenty-five microliters of sample were added to 25 μL of 400 μM 4MU-I substrate. The mixture was incubated at 37° C. for 30 minutes and 200 μL glycine carbonate buffer (pH 9.8) were added to quench the reaction. 4-Methylumbelliferone (4MU) (Sigma-Aldrich®, Saint-Louis, US) was used to prepare the standard calibration curve. Fluorescence was measured using a plate reader (TECAN® Infinite M1000, Mannedorf, Switzerland) with excitation at 355 nm and emission at 460 nm. IDUA enzyme production was expressed in units (μmol converted to product per minute) per sample volume (milliliters). The productivity was then calculated by dividing the enzyme production by the corresponding biomass concentration and the culture time and was expressed in percentage of the maximum productivity. The parameters KM, kcat and Vmax were calculated by linear fit on a Lineweaver-Burk plot (Ou et al., 2014; Mol. Genet. Metab. 111, 113-115).

1.7. Determination of Total Protein Concentration

Total protein concentration was quantified using the Bradford method based on the adsorption of the Bradford reagent on proteins. The complex reagent-protein leads to the change of light-absorption of the colorant which switch form red to blue, which absorbance is measurable at 595 nm. 50 μL of sample and 200 μL of Bradford reagent are mixed and incubated at room temperature during 10 min. Absorbance at 595 nm is then measured with a spectrometer. Bovine Serum Albumin was used as reference for the calibration curve. Total protein concentration (in mg/L) in the samples is then calculated.

2. Results

2.1. Effect of NaCl Washing on the Recovery of Secreted rIDUA

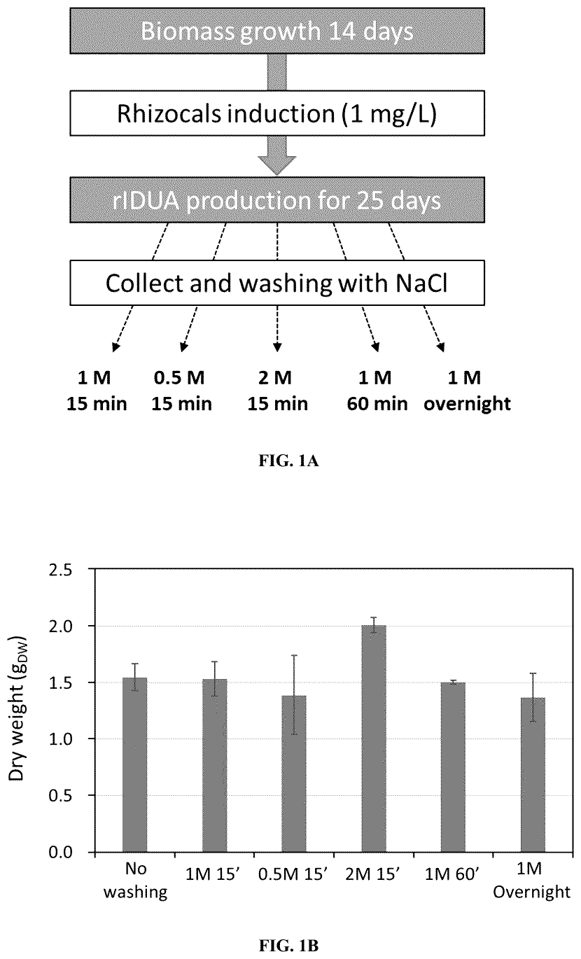

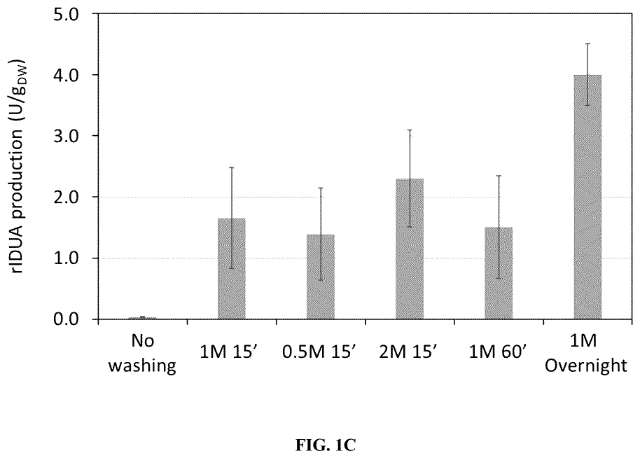

The effect of NaCl washing on the recovery of secreted rIDUA was assessed. At the end of the culture (14 days of culture as hairy roots and 25 days of culture after induction of rhizocals), the Brassica rapa rapa hairy roots were washed (i.e., incubated) with different concentrations of NaCl (0.5 M to 2 M) during different duration (15 min to overnight) and the rIDUA production was measured in the NaCl washing solution (FIG. 1A). As an internal control, the dry weight was measured and there is no variation in the different conditions assessed (FIG. 1B). An overnight washing with 1 M of NaCl allows to significantly increase by 2.5-fold the yield of recovery of active rIDUA with a low variability compare to other harvesting conditions tested (FIG. 1C). The other conditions also allow to recover a sufficient quantity of protein. Indeed, in the control culture condition where no washing with NaCl was performed, no rIDUA production was observed as compared to the production observed after 0.5M-2 M NaCl washing (see FIG. 1C). Altogether, these experiments illustrate the significant effect of a NaCl washing on the recovery of rIDUA produced in hairy roots.

2.2. E Feet of Stirring During the NaCl Washing Step on the Recovery of Secreted rIDUA

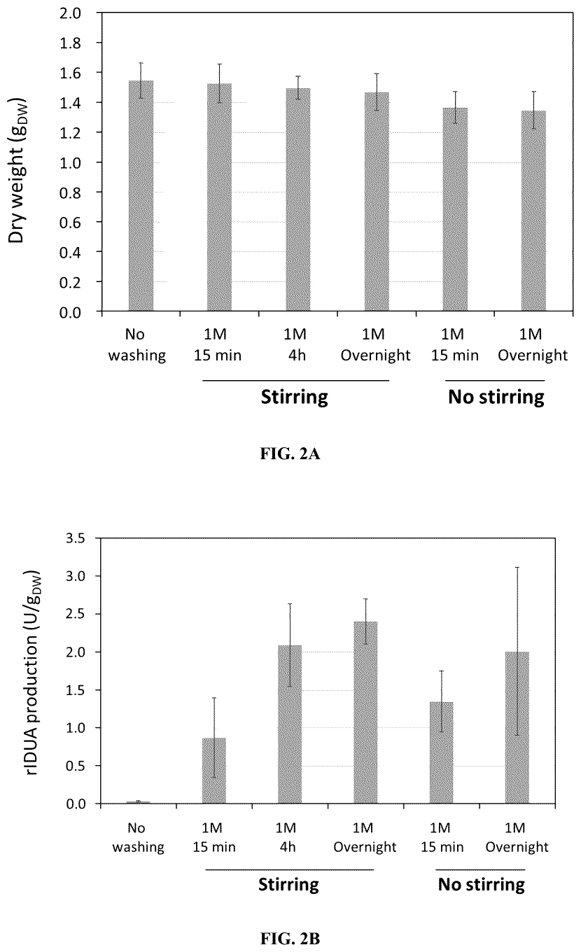

Experiments were performed to evaluate the effect of stirring (i.e., shaking) during the NaCl washing step. At the end of the culture (14 days of culture as hairy roots and 25 days of culture after induction of rhizocals), the Brassica rapa rapa hairy roots were washed (i.e., incubated) with 1 M of NaCl during different duration (15 min to overnight) with or without stirring (shaking at 40 rpm, using a shaking table) and the rIDUA production was measured in the NaCl washing solution. As an internal control, the dry weight was measured and there is no variation in the different conditions assessed (FIG. 2A). Regarding the rIDUA production, washing (i.e., incubating) the hairy roots with NaCl with or without stirring (i.e., shaking) did not significantly change the recovery of the protein of interest (FIG. 2B).

2.3. Effect of Using Different Salts During the Washing Step on the Recovery of Secreted rIDUA

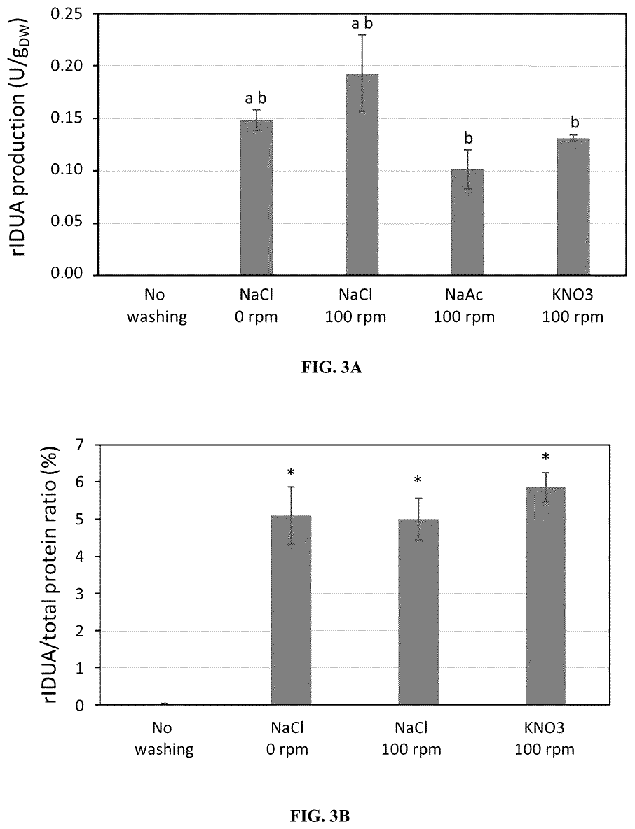

The effect of a washing (i.e., incubation) with an inorganic salt (sodium chloride (NaCl) or potassium nitrate (KNO₃)) or with an organic salt (sodium acetate (C₂H₃NaO₂)) on the recovery of secreted rIDUA was assessed. At the end of the culture (10 days of culture of the hairy roots followed by 10 days of culture after induction of rhizocals), Brassica napus hairy roots were washed (i.e., incubated) with different salts (NaCl, KNO₃ and C₂H₃NaO₂) at a concentration of 1M during 15 min, with or without stirring (shaking at 100 rpm, using a shaking table). The rIDUA production was then measured in the corresponding washing solutions (FIG. 3A).

First, the washing with NaCl allowed to significantly increase the yield of recovery of rIDUA in Brassica napus hairy roots as observed with Brassica rapa rapa hairy roots (see FIG. 1C). The data thus demonstrate that an increase in the yield of a secreted compound of interest can be obtained by washing (incubating) hairy roots with a solution comprising a salt. The effect of the salt washing on the yield of secreted compound is not specific to Brassica rapa rapa hairy roots.

Second, there was no significant difference of the yield of recovery of rIDUA in Brassica napus between the three salts tested. The data thus demonstrate that sodium chloride (NaCl), potassium nitrate (KNO₃), and sodium acetate (C₂H₃NaO₂) are equally effective in significantly increasing the yield of recovery of a secreted compound of interest in a hairy roots-based expression system.

Third, as previously observed for Brassica rapa rapa hairy roots in FIG. 2B, the stirring (i.e., shaking) had no impact on the yield of recovery of rIDUA in a Brassica napus hairy roots-based expression system. The data presented herein thus demonstrate that a step of washing (incubation) with a salt without any stirring (i.e., without any shaking) is sufficient to significantly increase the yield of recovery of a secreted compound of interest in a hairy roots-based expression system. Strikingly, an incubation of 15 mM without any stirring (i.e., without any shaking) is sufficient to significantly increase the yield of recovery of a secreted compound of interest in a hairy roots-based expression system.

The ratio of rIDUA/total protein was also assessed in the washing solutions after washing (incubating) of the Brassica napus hairy roots with sodium chloride (NaCl—with or without shaking at 100 rpm) or with potassium nitrate (KNO₃—with shaking at 100 rpm). Of note, the Bradford method for total protein quantification cannot be conducted in a solution comprising sodium acetate (C₂H₃NaO₂). As shown on FIG. 3B, the ratios were the same in all conditions of washing tested (NaCl with 0 and 100 rpm and KNO₃ with 100 rpm), supporting the fact that the hairy roots integrity is preserved.

2.4. Effect of the Presence of NaCl in the Culture Medium on the Recovery of Secreted rIDUA

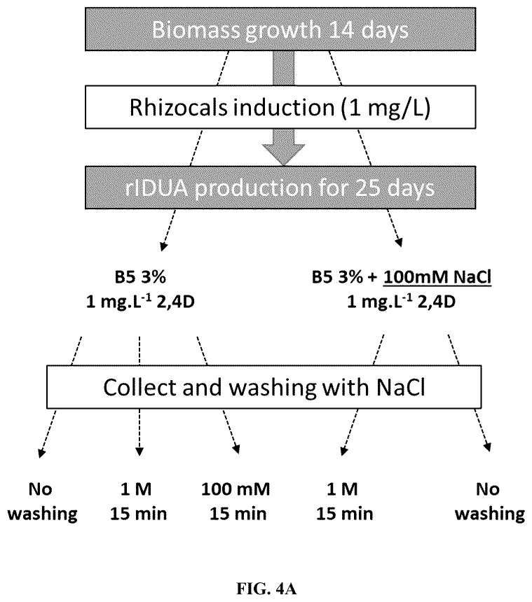

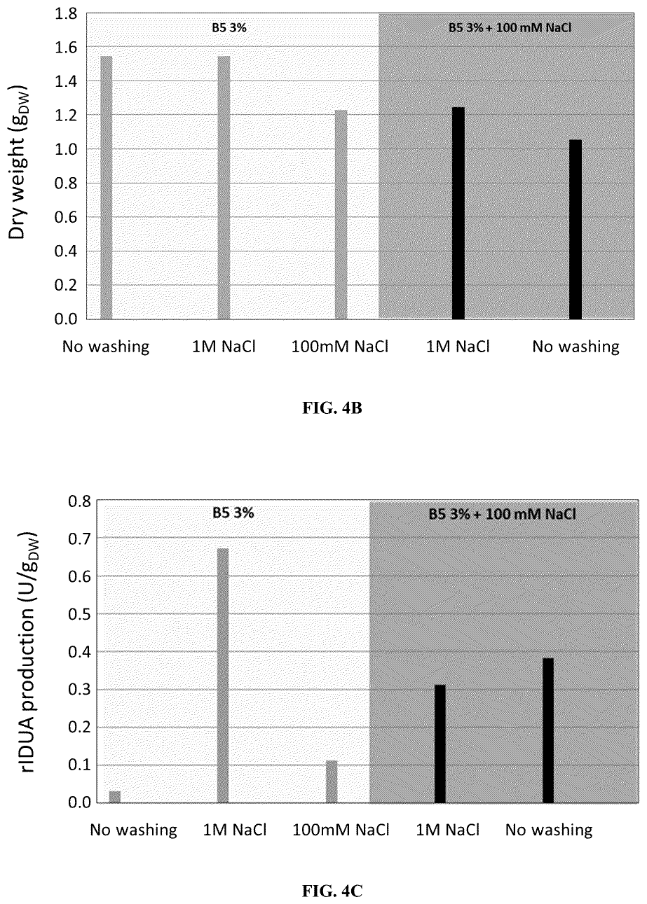

Further experiments were performed to evaluate whether the presence of NaCl in the culture medium has an impact or not on the recovery during the washing step with NaCl (1 M during 15 mM). Brassica rapa rapa hairy roots were cultured during 14 days and then rhizocals were induced and rIDUA was produced during 25 additional days of culture, which was performed with or without 100 mM of NaCl in the culture medium (FIG. 4A). As an internal control, the dry weight was measured and there is no major variation in the different conditions assessed (FIG. 4B). In the conditions where NaCl was present in the culture media, performing a washing with NaCl or no washing at all did not significantly affect protein recovery (FIG. 4C—right panel). In the condition where NaCl was absent from the culture media, a significantly higher quantity of protein was recovered after washing the hairy roots with 1 M NaCl as compared to either washing with 100 mM of NaCl or with no washing at all (FIG. 4C—left panel). Washing with 1 M NaCl following culture in the absence of NaCl increases by 2-fold the protein recovery yield as compared to washing with 1 M NaCl following culture in the presence of 100 mM NaCl. Of note, washing with 1 M NaCl following culture in the absence of NaCl also significantly increases the protein recovery yield as compared to culturing in the presence of 100 mM not followed by any washing.

Taken together, the data presented in FIG. 4C thus demonstrate that washing (i.e., incubating) the hairy roots with 1 M NaCl at the end of the culture significantly improves the protein recovery yield, as compared to using 100 mM NaCl either during the culture or in a washing step at the end of a culture carried out without NaCl. The data also demonstrate that washing the hairy roots with 1 M NaCl allows a significantly higher recovery of protein after a culture carried out in the absence of NaCl as compared to a culture carried out in the presence of 100 mM NaCl.

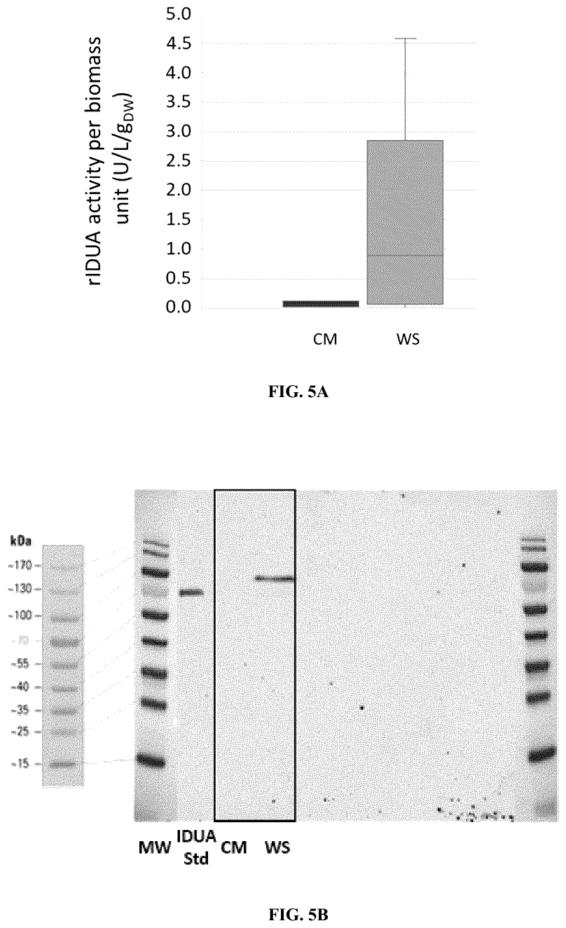

Finally, the effect of NaCl washing on the recovery of secreted rIDUA was assessed in different clones. At the end of the culture, the Brassica rapa rapa hairy roots were washed with 1 M NaCl during 15 mM and the rIDUA production as well as the quantification of rIDUA protein were measured both in the culture medium (CM) and in the NaCl washing solution (WS). As presented in FIGS. 5A and 5B, the washing with NaCl allows to recover a much higher quantity of protein in comparison with the condition without washing.

### Example 2: Continuous Production and Recovery of Secreted eGFP

1. Material and Methods

1.1. Molecular Construction

The molecular construction is characterized by the presence of a 35S double promoter, a signal peptide (SP) coding sequence from Arabidopsis pectin methylesterase (PME) At1g69940, the nucleic acid encoding the recombinant protein with a tag (eGFP) and a CaMV polyA sequence (Huet et al., 2014; Biotechnol Lett, 36(1), pp. 181-90). The selected hairy root clone Ver2 ml was developed by Pr Francois Guerineau and Pr Michèle Boitel-Conti from BIOPI's lab (Biologie des plantes et innovations) at the University of Picardie Jules Verne (UPJV).

1.2. Culture of Hairy Roots

Eighteen flasks were seeded with Brassica rapa rapa hairy roots Ver2 ml (10 gFW/L—gram of fresh weight per liter) in B5 medium and grown for 14 days. After this first phase, the rhizocal induction was carried out by changing the culture medium and by adding 1 mg/L of 2,4 D, in order to enhance the production of recombinant protein. After a first production cycle of 14 days, cultures were washed aseptically with a sterile solution of NaCl at 1 M during 15 min, then rinsed with sterile ultrapure water and finally transferred into fresh culture medium in order to begin a new production cycle. Two cycles of production phase were performed, with the hairy roots collected and washed to recover eGFP. Two cultures were also collected as reference for the end of the production cycle, in order to measure biomass.

1.3. Collect and Protein Recovery

At the end of a protein production process by hairy roots, culture medium was systematically harvested and hairy roots were washed with a sodium chloride solution (1 M) for 15 minutes under gentle shaking (100 rpm). Samples of washing solution were collected, hairy roots were rinsed with ultrapure water and then dried at for 24 h for dry weight determination and eGFP concentration was determined.

1.4. Determination of eGFP Concentration

The concentration of eGFP in Brassica rapa rapa hairy root culture medium collected from transformed hairy roots cultures was determined by using a fluorimetric method. Indeed, GFP is naturally fluorescent at excitation and emission wavelengths of 490 nm and 510 nm respectively. Fluorescence of the culture medium was measured using a plate reader (TECAN® Infinite M1000, Mannedorf, Switzerland) with excitation at 490 nm and emission at 510 nm. The standard curve was prepared using a stock culture medium containing eGFP at a concentration of 102 mg/L (previously determined using purified eGFP, Interchim, ref c8T3651) and preserved at −20° C.

2. Results

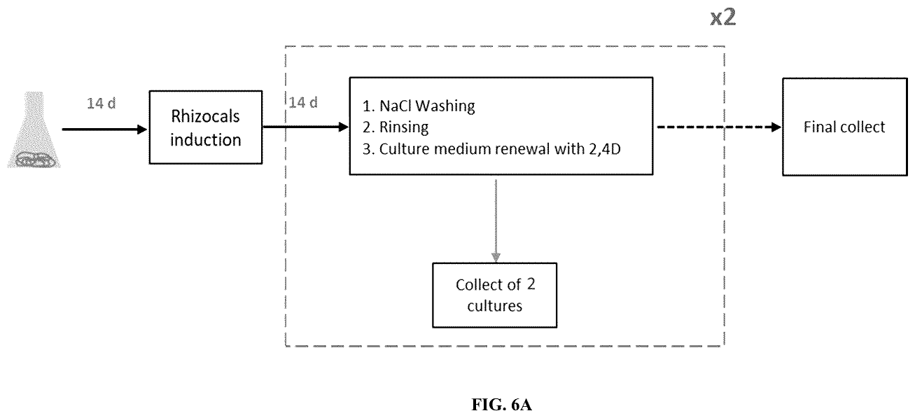

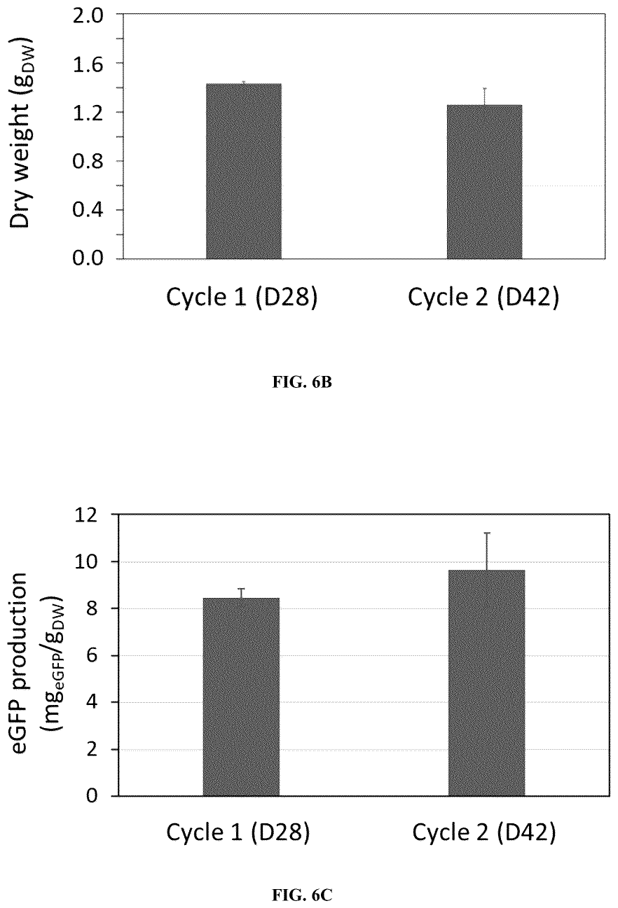

Ver2 ml hairy roots were cultured during 14 days and then during 14 additional days after the induction of rhizocals and eGFP protein was recovered upon a washing step consisting of addition of a salt solution of NaCl at 1 M and letting the salt solution contact the hairy roots during 15 min After the washing step (incubating step), the hairy roots are used again for a new cycle of production during 14 days and washed (incubated) again with NaCl (FIG. 6A). As represented in FIGS. 6B and 6C, the continuous system allows to recover equivalent quantities of eGFP after the first and the second cycle.

1-13. (canceled)

14. A method for recovering a secreted compound of interest in a hairy roots-based expression system, the method comprising incubating the hairy roots in a solution comprising from about 250 mM to about 4 M of a salt.

15. The method according to claim 14, wherein the hairy roots-based expression system comprises a phase of culture wherein the compound of interest is produced by the hairy roots in a culture medium.

16. The method according to claim 15, wherein the compound of interest is produced in the presence of an inductor of rhizocals.

17. The method according to claim 14, wherein the salt is organic or inorganic.

18. The method according to claim 14, wherein the salt is selected from a group consisting of sodium (Na) salt, potassium (K) salt, chloride (Cl) salt, sulfate (SO₄) salt, nitrate (NO₃) salt, ammonium (NH₄) salt, phosphate (PO₄) salt, and any combination thereof.

19. The method according to claim 14, wherein the salt consists in sodium chloride (NaCl), potassium chloride (KCl), potassium nitrate (KNO₃), sodium sulfate (Na₂SO₄), potassium phosphate (KH₂PO₄), ammonium sulfate (NH₄) 2 SO₄, ammonium nitrate (NH₄NO₃), sodium carbonate (Na₂CO₃), sodium glutamate (C₅H₈NNaO₄), sodium citrate (Na₃C₆H₅O₇), and/or sodium acetate (C₂H₃NaO₂).

20. The method according to claim 14, wherein the solution of salt comprises from about 500 mM to about 2.5 M of salt.

21. The method according to claim 14, wherein the solution of salt comprises about 1M of salt.

22. The method according to claim 14, wherein the hairy roots are incubated in the solution comprising the salt for about 5 mM to about 24 h.

23. The method according to claim 14, wherein the hairy roots are incubated in the solution comprising the salt for about 15 mM to about 18 h.

24. The method according to claim 15, wherein the culture medium comprises less than 250 mM of salt.

25. The method according to claim 15, wherein the culture medium comprises less than 250 mM of salt selected from a group consisting of a sodium (Na) salt, a potassium (K) salt, a chloride (Cl) salt, sulfate (SO₄) salt, nitrate (NO₃) salt, ammonium (NH₄) salt, phosphate (PO₄) salt, or any combination thereof.

26. The method according to claim 14, wherein said hairy roots belong to the species Brassica rapa rapa, Brassica napus, Salvia Milthiorrhiza, Panax Ginseng, Armoracia rusticana, Trigonella foenumgraceum, Lippia dulcis, Lithospermum erythrorhizon, Ophiorrhiza pumila, and Echinacea purpurea, Echinacea Angustifolia, Puerariaphaseoloides, Harpagophytum Procumbens, Morinda Citrifolia, Hypericum Perforatum, Derris trifolia, Salvia miltiorrhiza, Salvia prevalzkii, Echinacea pallida, Cistanche tubulosa, Glycyrrhiza glabra, Sophora flavescens, Rhodiola Rosea, Polygonum cuspidatum, Fallopia multiflora, Lepidium peruvianum, Whitania Somnifera, Astragalus Membranaceous, Berberis Vulgaris, Sanguinaria canadensis, Eleutherococcus Senticosus, Cannabis sativa, Hydrastis Canadensis, Arctium Majus, Piper methysticium, Pueraria lobata, Glycyrrhiza uralensis, Ptychopetalum olacoides, Dioscorea Vollosa, Yucca shidigera, Panax quinquefolius, Azadirachta indica, Catharanthus trichophyllus, Calystegia sepium, Atropa belladonna, Hyoscyamus muticus, Artemisia annua, Datura stramonium, Arabidopsis thaliana, Stizolobium, Hassjoo, Ipomea aquatica, Perilla fruitescnens, Catharanthus roseus, Taxus brevifolia, Gloriosa Superba, Saponaria officinalis, Solanum tuberosum, Nicotiana tabacum, Nicotiana benthamiana or Cinchosa Pubescens.

27. The method according to claim 14, wherein said hairy roots belong to the Brassica rapa rapa or Brassica napus species.

28. The method according to claim 14, wherein said compound of interest is selected from the group consisting of metabolites, non-peptidic hormones and recombinant proteins.

29. The method according to claim 28, wherein the recombinant protein is selected from the group consisting of allergens; vaccines; enzymes; enzyme inhibitors; antibodies; antibody fragments; antigens, toxins; anti-microbial peptides; peptidic hormones; growth factors; blood proteins; receptors and/or signaling proteins; protein component of biomedical standards; protein component of cell culture media; fusion and/or tagged proteins; cysteine (disulfide bridges)-rich peptides and proteins; and plant proteins.

30. The method according to claim 28, wherein the recombinant protein is a plant protein selected from the group comprising lectins and papain.

31. The method according to claim 28, wherein the metabolites are selected from the group consisting of polyphenols, alkaloids, cannabinoids, terpenoids, steroids, flavonoids, and tannins.

32. A method for the continuous production of a secreted compound of interest by a hairy roots-based expression system, the method comprising:
a) the production of a compound of interest by hairy roots in a culture medium;
b) the removal of the culture medium;
c) the recovery of the secreted compound of interest by incubating the hairy roots in a solution comprising from about 250 mM to about 4 M of a salt;
d) optionally, the rinsing of the hairy roots; and
e) the addition of a fresh culture medium to the hairy roots as treated in step c) or d), wherein steps a) through e) are sequentially repeated between about 1 and about 5 times.

33. The method according to claim 32, wherein the culture medium is suitable for rhizocals induction.
